(19) United States
(12) Patent Application Publication
(10) Pub. No.: US 2026/0364361 A1
(43) Pub. Date: Nov. 19, 2026

(54) MULTI-DIRECTIONAL ENERGY HARVESTER FOR IMPLANTABLE DEVICES

(71) Applicant: Medtronic, Inc., Minneapolis, MN (US)

(72) Inventors: Winton J. Parker, Melrose, MA (US); Anthony W. Olivett, Brooklyn Center, MN (US); Amin Karami, Ann Arbor, MI (US); Kevin L. Sack, Minneapolis, MN (US); Can Cinbis, Salt Lake City, UT (US)

(21) Appl. No.: 19/476,095

(22) PCT Filed: Mar. 19, 2024
(86) PCT No.: PCT/IB2024/052637

§ 371 Date: Oct. 16, 2025

Related U.S. Application Data
(60) Provisional application No. 63/496,471, filed on Apr. 17, 2023.

(51) Int. Cl. A61N 1/378; A61N 1/362; A61N 1/375; H02J 50/00; H02J 105/46; H02N 2/18

(52) U.S. Cl. CPC A61N 1/378; A61N 1/362; A61N 1/37512; A61N 1/3756; H02J 50/001; H02N 2/188; H02J 2105/46

(57) ABSTRACT

Energy harvesting mechanisms for implantable devices are provided. In some embodiments, a device includes a housing configured to be implanted within a patient. The housing can have a proximal end, a distal end, and a longitudinal axis extending between the proximal end and the distal end. The device can also include a power source positioned within the housing, and an energy harvesting mechanism positioned within the housing and configured to charge the power source. The energy harvesting mechanism can include a plurality of piezoelectric elements that are aligned with the longitudinal axis of the housing. Each piezoelectric element can face a different direction.

This application claims the benefit of U.S. Provisional Patent Application Ser. No. 63/496,471, filed 17 Apr. 2023, the entire content of which is incorporated herein by reference.

### TECHNICAL FIELD

The present technology generally relates to medical devices, and in particular, to multi-directional energy harvesters for implantable devices.

### BACKGROUND

Various types of implantable medical devices have been developed for monitoring or treating one or more conditions of a patient. For example, a cardiac pacemaker can monitor a patient's heart activity and provide therapeutic electrical stimulation to the heart via electrodes. The electrical stimulation provided by the cardiac pacemaker can include signals such as pacing pulses to address abnormal cardiac rhythms (e.g., bradycardia). Some types of cardiac pacemakers are implanted a distance from the heart and are coupled to one or more leads that extend intravascularly into the heart to position the electrodes in contact with cardiac tissue. However, the leads may be prone to fracture, which may result in unreliable or incorrect pacing, and may require replacement of the lead or even the entire pacemaker.

Some types of cardiac pacemakers are sized to be completely implanted within one of the chambers of the heart, and may include electrodes integrated with or attached to the device housing rather than leads. Such pacemakers can be less invasive than traditional pacemakers and can avoid complications associated with lead fracture. However, the relatively small size of such pacemakers may limit the types of power sources that can be incorporated into the device.

### BRIEF DESCRIPTION OF THE DRAWINGS

Many aspects of the present disclosure can be better understood with reference to the following drawings. The components in the drawings are not necessarily to scale. Instead, emphasis is placed on illustrating clearly the principles of the present disclosure.

FIG. 1 illustrates a pacing device implanted in the heart of a patient, in accordance with embodiments of the present technology.

FIG. 2 is a perspective view of a pacing device configured in accordance with embodiments of the present technology.

FIG. 3 is a side view of another pacing device configured in accordance with embodiments of the present technology.

FIG. 4 is a schematic block diagram illustrating electronic components of a pacing device configured in accordance with embodiments of the present technology.

FIG. 5A is a side cross-sectional view of a device including an energy harvesting mechanism, in accordance with embodiments of the present technology.

FIG. 5B is a perspective view of the energy harvesting mechanism of FIG. 5A.

FIG. 5C is a partially schematic illustration of the energy harvesting mechanism of FIG. 5A in a flattened configuration.

FIG. 5D is an end cross-sectional view of the energy harvesting mechanism of FIG. 5A.

FIG. 6 is a partially schematic end view of an energy harvesting mechanism in a plurality of different orientations, in accordance with embodiments of the present technology.

FIG. 7A is a partially schematic illustration of an energy harvesting mechanism in a flattened configuration, in accordance with embodiments of the present technology.

FIG. 7B is an end cross-sectional view of the energy harvesting mechanism of FIG. 7A.

FIG. 8A is a side cross-sectional view of a device including an energy harvesting mechanism, in accordance with embodiments of the present technology.

FIG. 8B is a perspective view of the energy harvesting mechanism of FIG. 8A.

FIG. 8C is a top view of the energy harvesting mechanism of FIG. 8A.

FIG. 8D is a side view of the energy harvesting mechanism of FIG. 8A.

FIG. 8E is an end view of the energy harvesting mechanism of FIG. 8A.

FIG. 9 is a perspective view of an energy harvesting mechanism configured in accordance with embodiments of the present technology.

### DETAILED DESCRIPTION

The present technology relates to energy harvesters for implantable devices. In some embodiments, for example, a device includes a housing configured to be implanted within a patient (e.g., within a single chamber of the patient's heart). The housing can have a proximal end, a distal end, and a longitudinal axis extending between the proximal end and the distal end. The device can include a power source positioned within the housing to power various operations (e.g., providing electrical stimulation to the patient). The device can also include an energy harvesting mechanism positioned within the housing and configured to charge the power source. The energy harvesting mechanism can include a plurality of piezoelectric elements that are aligned with the longitudinal axis of the housing, and that generate energy when mechanically deflected by physiological motion of the patient (e.g., cardiac motion). In some embodiments, the piezoelectric elements are arranged to face different directions and/or have different principal bending directions, thus providing efficient energy harvesting for many different input motion directions.

The present technology can provide numerous advantages compared to conventional approaches for powering implantable devices. For example, the use of kinetic energy harvesters that produce electrical energy from physiological motion as described herein can extend the lifetime of the implantable device by allowing for recharging in situ within the patient's body. However, conventional energy harvesters frequently exhibit directional dependency, in that the power output of the harvester is significantly affected by the orientation of the harvester relative to the direction of the motion. For example, piezoelectric beams typically generate maximum power when the direction of the motion is perpendicular to the plane of the beam, thus resulting in maximum deflection of the beam. If the beam is not properly aligned with the direction of motion (e.g., the direction of motion is parallel to the plane of the beam), the beam may exhibit little or no deflection, thus resulting in negligible power output. However, physiological motion is often multidirectional and can vary over time (e.g., depending on the patient's posture, activity, anatomy, etc.), such that it can be difficult or impossible to predict the optimal alignment of the implantable device for energy harvesting purposes.

To address these and other challenges, the present technology provides implantable devices that are configured to harvest energy from multiple directions of motion with high efficiency. In some embodiments, the energy harvesting mechanisms herein include multiple piezoelectric elements that are geometrically arranged so that the power output of each element is enhanced (e.g., maximized) for a different direction of motion. Stated differently, each piezoelectric element can be oriented in a different direction, such that for any direction of motion, at least one of the piezoelectric elements is “active” and exhibits significant deflection. This approach can increase the robustness of the energy harvesting mechanism to different directions of motion, thus increasing the longevity of the implantable device and/or broadening the potential inclusion criteria for suitability. Additionally, the present technology allows the implant procedure to be agnostic to the alignment of the device, which can reduce the complexity and duration of the implant procedure. Moreover, the embodiments herein can use multiple piezoelectric elements arranged in a folded and/or overlapping configuration to decrease space requirements while also maintaining a low resonant frequency compatible with the frequencies present in physiological motion, thus allowing the energy harvesting mechanism to be used in implantable devices with strict size constraints (e.g., devices implanted in a single heart chamber).

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

The headings provided herein are for convenience only and do not interpret the scope or meaning of the claimed present technology. Embodiments under any one heading may be used in conjunction with embodiments under any other heading.

### I. Overview of Implantable Pacing Devices

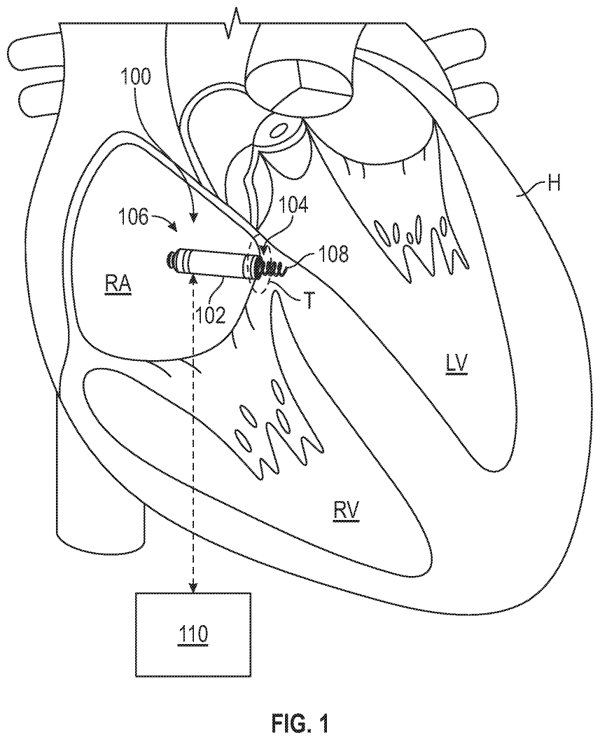

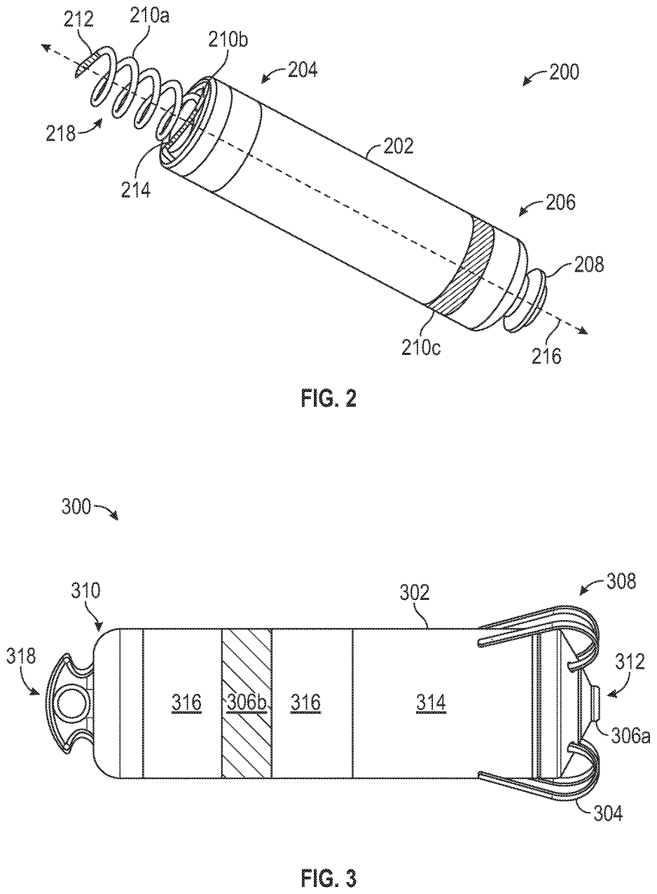

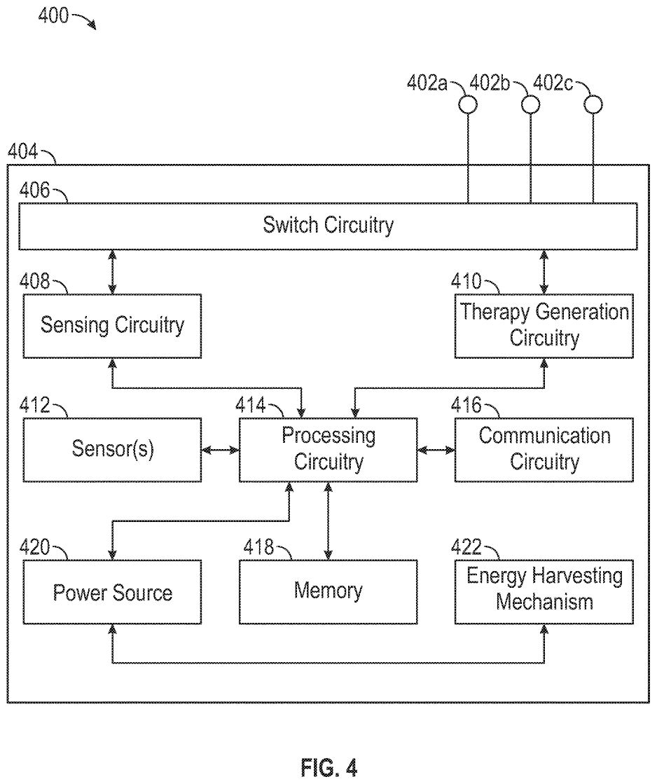

FIGS. 1-4 provide a general overview of implantable devices configured in accordance with embodiments of the present technology. Specifically, FIG. 1 illustrates a pacing device implanted in a patient's heart, FIG. 2 illustrates an example configuration for a pacing device, FIG. 3 illustrates another example configuration for a pacing device, and FIG. 4 illustrates electronic components that can be included in a pacing device. Any of the features of the embodiments of FIGS. 1-4 can be combined with each other and/or with any of the other embodiments described herein.

Referring first to FIG. 1, which illustrates a pacing device 100 implanted in the heart H of a patient, the device 100 is configured to monitor activity of the heart H and provide electrical stimulation (e.g., pacing signals) to the heart H. In some embodiments, the device 100 is a leadless intracardiac pacemaker configured to be implanted entirely within a heart chamber, such as entirely within the right atrium (RA), entirely within the right ventricle (RV), entirely within the left atrium (LA), or entirely within the left ventricle (LV). The device 100 can be implanted at any of a variety of locations to sense and/or deliver therapy to any chamber or chambers of the heart H. For example, as shown in FIG. 1, the device 100 can be a right atrial intracardiac pacemaker that is implanted in the RA of the patient's heart H in a target implant region T (e.g., the triangle of Koch). The target implant region T can lie between the bundle of His and the coronary sinus, and/or can be adjacent to the tricuspid valve. In other embodiments, the device 100 can instead be configured as a right ventricular intracardiac pacemaker that is implanted in the RV of the heart H, with the target implant region T lying along the endocardial wall at or near the apex of the RV.

The device 100 can include a housing 102 having a size and form factor suitable for transvenous delivery into the heart H via a catheter. In the illustrated embodiment, the housing 102 has an elongate shape extending from a distal portion 104 to a proximal portion 106. The housing 102 can have a generally cylindrical shape (e.g., pill-shaped or capsule-shaped), a generally prismatic shape (e.g., a rectangular prism), or any other suitable shape. The housing 102 can define an interior cavity that contains the electronic components of the device 100 (e.g., circuitry, power source, sensors).

The device 100 can include a fixation mechanism 108 to secure the device 100 to the tissue of the heart H. For example, the fixation mechanism 108 can include one or more fixation elements configured to penetrate into tissue, such as one or more tines, coils, barbs, etc. In the illustrated embodiment, the fixation mechanism 108 is coupled to and extends outwardly from the distal portion 104 of the housing 102. Accordingly, when the device 100 is implanted, the distal portion 104 can be positioned in contact with or in close proximity to the cardiac tissue, while the proximal portion 106 can be spaced apart from the cardiac tissue. In other embodiments, however, the fixation mechanism 108 can be located at a different portion of the device 100.

The device 100 also includes a plurality of electrodes configured to sense electrical activity of the heart H and/or deliver electrical therapy to the heart H. For example, the device 100 can include two, three, four, five, six, seven, eight, nine, ten, or more electrodes. Each electrode can be positioned at any suitable portion of the device 100, such as on or coupled to the housing 102 (e.g., the distal portion 104, the proximal portion 106, an intermediate location between the distal portion 104 and proximal portion 106), or on or coupled to the fixation mechanism 108. In some embodiments, the device 100 includes one or more electrodes (e.g., cathodes) that directly contact the cardiac tissue (e.g., of a single heart chamber or multiple heart chambers) to sense the activity thereof and/or deliver electrical therapy thereto. Such electrode(s) can be located at the distal portion 104 of the housing 102 and/or incorporated into the fixation mechanism 108, for example. The device 100 can also include at least one electrode (e.g., an anode and/or return electrode) that does not directly contact cardiac tissue. Such electrode(s) can be located at portions of the housing 102 that are spaced apart from cardiac tissue, such as the proximal portion 106. Optionally, a single electrode may serve as a cathode for certain operations, and may serve as an anode and/or return electrode for other operations.

In some embodiments, the device 100 is operably coupled to an external device 110 shown schematically) via bidirectional wireless communication, such as BLUETOOTH®, Wi-Fi, Medical Implant Communication Service (MICS), or other radiofrequency communication technique. The external device 110 can be a computing device or system that is located outside of the patient's body, and can be used in a healthcare setting (e.g., in a clinic, hospital or other medical facility), at the patient's home, or suitable combinations thereof. The external device 110 can be configured to control various operational parameters of the device 100, such as therapy parameters (e.g., pacing control parameters such as pacing interval), sensing parameters, power management parameters, etc. For instance, the external device 110 can transmit control signals to the device 100 to program one or more operational parameters of the device 100. Optionally, the external device 110 can display information relating to and/or received from the device 100, such as intracardiac electrogram (EGM) signals obtained by the device 100, motion sensor signals acquired by the device 100, operational parameters of the device 100, etc. In some embodiments, the external device 110 transmits information received from the device 100 to another computing device or system (e.g., a computer, laptop, workstation, mobile device, server, remote patient management system) for display, processing, and/or storage, using any suitable wired or wireless communication technique. The external device 110 can serve as a “programmer” that allows a physician, patient, or other individual to monitor and/or control the operations of the device 100.

Although FIG. 1 illustrates a single device 100, the present technology is also applicable to implantable systems including multiple devices 100 implanted at different locations in the heart H. For example, an implantable system can include a first device 100 in the RA and a second device 100 in the RV. In such embodiments, each device 100 can independently have any of the features described herein.

FIG. 2 is a perspective view of a pacing device 200 configured in accordance with embodiments of the present technology. The device 200 is configured to be implanted within a chamber of a heart of the patient to monitor activity of the heart and/or provide electrical therapy (e.g., pacing therapy) to the heart. The device 200 includes a housing 202 having a size and form factor that allows the device 200 to be entirely implanted within a single chamber of the patient's heart. In the illustrated embodiment, the housing 202 has an elongate shape (e.g., a generally cylindrical shape, a generally prismatic shape) extending between a distal end 204 and proximal end 206. The housing 202 can define a hermetically sealed internal cavity for housing the electronic components of the device 200. The housing 202 can also include an attachment mechanism 208 (e.g., at the proximal end 206) configured to temporarily engage with a delivery tool during implantation and/or extraction of the device 200.

The housing 202 can be formed partially or entirely from a conductive material, such as titanium or titanium alloy, stainless steel, MP35N (a non-magnetic nickel-cobalt-chromium-molybdenum alloy), a platinum alloy, or other biocompatible metal or metal alloy, or other suitable conductive material. Alternatively or in combination, the housing 202 can be formed partially or entirely from a nonconductive (e.g., insulative) material, such as ceramic, glass, sapphire, silicone, polyurethane, epoxy, acetyl co-polymer plastics, polyether ether ketone (PEEK), a liquid crystal polymer, other biocompatible polymer, or other suitable nonconductive material.

The device 200 can include a plurality of electrodes 210a-210c configured to sense electrical activity of the heart and/or deliver electrical stimulation to the heart. In the illustrated embodiment, for example, the device 200 includes a first electrode 210a and a second electrode 210b at or proximate to the distal end 204 of the housing 202, and a third electrode 210c on the housing 202. The first and second electrodes 210a, 210b can be configured as cathode electrodes that directly contact cardiac tissue, e.g., a distal end of the first electrode 210a can be configured to rest within a ventricular myocardium of the patient, and the second electrode 210b can be configured to contact an atrial endocardium of the patient. The third electrode 210c can be configured as an anode and/or return electrode that does not directly contact cardiac tissue.

As shown in FIG. 2, the first electrode 210a can be an elongate structure that extends from the distal end 204 of the housing 202 to penetrate through the wall tissue of a first heart chamber (e.g., the chamber in which the device 200 is implanted) into wall tissue of a second, different heart chamber. For example, in some embodiments, the device 200 is implanted in the RA with the distal end 204 oriented toward the LV (e.g., similar to the arrangement of the device 100 in FIG. 1), and the first electrode 210a extends through the wall tissue of the RA and into the wall tissue of the LV. In the illustrated embodiment, the first electrode 210a is configured as a coil (e.g., a helical and/or spiral coil), while in other embodiments, the first electrode 210a can have a different form factor (e.g., an elongate dart, barb, tine, or other tissue penetrating element). The first electrode 210a can include a proximal end that is coupled to the distal end 204 of the housing 202, and a free distal end that is not attached to the housing 202. The distal end of the first electrode 210a can have a conical, hemi-spherical, or slanted edge distal tip with a narrow tip diameter (e.g., less than 1 mm) for penetrating into and through tissue layers. In some embodiments, the distal end of the first electrode 210a can have a sharpened or angular tip, and/or sharpened or beveled edges, but the degree of sharpness can be constrained to avoid a cutting action that could lead to lateral displacement of the distal end of the first electrode 210a and undesired tissue trauma.

The second electrode 210b can be a structure that extends from the distal end 204 of the housing 202 to contact the wall tissue of the first heart chamber without penetrating the wall tissue. The second electrode 210b can be located proximal to the first electrode 210a. The second electrode 210b can be configured as a coil (e.g., a partial helical and/or spiral coil that does not form a full turn), loop, button, pad, or any other suitable form factor. The second electrode 210b can include a proximal end that is coupled to the distal end 204 of the housing 202, and a distal end that may or may not be coupled to the housing 202. In some embodiments, the second electrode 210b is configured to flexibly maintain contact with wall tissue of the heart chamber in which the device 200 is implanted, (e.g., the RA endocardium), despite variations in the tissue surface and/or in the distance between the distal end 204 of the housing 202 and the tissue surface, which may occur as the wall tissue moves during the cardiac cycle. Accordingly, the second electrode 210b can be flexible and/or have spring-like properties, e.g., the second electrode 210b can have a spring bias that urges at least a portion of the second electrode 210b away from the distal end 204 of the housing 202 and toward the wall tissue of the heart chamber to maintain consistent contact.

The first and second electrodes 210a, 210b can each be formed of an electrically conductive material, such as titanium, platinum, iridium, tantalum, or alloys thereof. The first electrode 210a can include one or more insulative coatings (e.g., parylene, polyurethane, silicone, epoxy) that reduce the electrically conductive surface area of the first electrode 210a to define a first electrically active region 212 (e.g., at or near the distal end of the first electrode 210a). The second electrode 210b can include one or more insulative coatings (e.g., parylene, polyurethane, silicone, epoxy) that reduce the electrically conductive surface area of the second electrode 210b to define a second electrically active region 214 (e.g., at an intermediate region between the proximal and distal ends of the second electrode 210b). This approach can increase the electrical impedance of the first and second electrodes 210a, 210b, and thereby reduce the current delivered during a pacing pulse, which can conserve the power used by the device 200. In some embodiments, the first and second electrodes 210a, 210b include an electrically conductive material coating (e.g., TiN) on the first and second electrically active regions 212, 214, respectively, to define the active regions. The first and second electrodes 210a, 210b can be made of the same materials, or can be made of different materials.

All, substantially all, or a portion of the housing 202 can serve as a third electrode 210c (e.g., an anode and/or return electrode) during pacing and/or sensing. In some embodiments, the third electrode 210c partially or fully circumscribes a portion of the housing 202 at or near the proximal end 206. Although FIG. 2 illustrates the third electrode 210c as a singular band, in other embodiments, the third electrode 210c can include multiple segments spaced a distance apart along a longitudinal axis 216 of the housing 202 and/or around a perimeter of the housing 202. Additionally, the third electrode 210c can also be located at other positions along the housing 202, e.g., located at or near the distal end 204 or at other positions along the longitudinal axis 216.

In embodiments where the housing 202 is formed from a conductive material, one or more portions of the housing 202 can be electrically insulated by a nonconductive material, such as a coating of parylene, polyurethane, silicone, epoxy or other biocompatible polymer, or other suitable material. For the portions of the housing 202 without the nonconductive material, one or more discrete areas of the housing 202 with conductive material can be exposed to define the third electrode 210c. In embodiments where the housing 202 is formed from a nonconductive material, a conductive material can be applied to one or more discrete areas of the housing 202 to form the third electrode 210c. Optionally, the third electrode 210c can be a discrete component (e.g., a ring electrode) that is coupled to the housing 202.

The electrodes 210a-210c can be used to sense electrical activity of one or more heart chambers and/or to deliver electrical stimulation to one or more heart chambers. For example, the first electrode 210a can be paired with the second electrode 210b or the third electrode 210c to for sensing ventricular signals and delivering ventricular pacing pulses. As another example, the second electrode 210b can be paired with the first electrode 210a or the third electrode 210c for sensing atrial signals and delivering pacing pulses to the atrial myocardium. In a further example, the third electrode 210c can be paired at different times with both the first electrode 210a and the second electrode 210b for either ventricular or atrial functionality, respectively. As yet another example, the first electrode 210a and the second electrode 210b can be paired with each other with different polarities for atrial and ventricular functionality.

In some embodiments, the second electrode 210b is configured as an atrial cathode electrode for delivering pacing pulses to the atrial tissue at a target implant region in combination with the third electrode 210c. The second electrode 210b and the third electrode 210c can also be used to sense atrial P-waves for use in controlling atrial pacing pulses (e.g., delivered in the absence of a sensed P-wave) and for controlling atrial-synchronized ventricular pacing pulses delivered using the first electrode 210a as a cathode and the third electrode 210c as the return anode. The configuration of the electrodes 210a-210c illustrated in FIG. 2 allows the device 200 to sense cardiac signals from and/or deliver cardiac pacing to one or more chambers of the heart. For example, the present technology can facilitate the delivery of A-V synchronous pacing using a single device 200 implanted within a single heart chamber (e.g., the RA).

The device 200 can include a fixation mechanism 218 configured to fix the device 200 to cardiac tissue at a target implant region (e.g., the triangle of Koch). In the illustrated embodiment, the first electrode 210a and/or second electrode 210b at the distal end 204 of the housing 202 can serve as the fixation mechanism 218. In other embodiments, the fixation mechanism 218 can be a different component than the first electrode 210a and/or the second electrode 210b, such one or more separate barbs, tines, coils, darts, etc.

FIG. 3 is a side view of another pacing device 300 configured in accordance with embodiments of the present technology. The device 300 is configured to be implanted within a chamber of a heart of a patient to monitor activity of the heart and/or to provide electrical therapy to the heart. In the embodiment shown in FIG. 3, the device 300 includes a housing 302, a plurality of fixation tines 304, a first electrode 306a, and a second electrode 306b.

The housing 302 can have a size and form factor that allows the device 300 to be entirely implanted within a chamber of a heart of a patient. For example, as shown in FIG. 3, the housing 302 has a generally cylindrical (e.g., pill-shaped or capsule-shaped), elongate form factor extending between a distal end 308 and a proximal end 310. The housing 302 contains electronic components of the device 300, and can be hermetically or near-hermetically sealed to prevent fluid ingress into the housing 302. The materials used to form the housing 302 can include any of the conductive and nonconductive materials described above with respect to FIG. 2.

The device 300 can include a fixation mechanism configured to fix the device 300 to cardiac tissue at a target implant region (e.g., the endocardial wall near the apex of the RV). In the illustrated embodiment, the device 300 includes a plurality of fixation tines 304 extending from the distal end 308 of the housing 302 and configured to engage with cardiac tissue to secure the housing 302 at a fixed position within the chamber of the heart. The fixation tines 304 can be configured to anchor the housing 302 to the cardiac tissue such that the device 300 moves along with the cardiac tissue during cardiac contractions. The device 300 can include any suitable number of fixation tines 304, such as one, two, three, four, five, or more fixation tines 304. The fixation tines 304 can be fabricated from any suitable material, such as a shape memory material (e.g., Nitinol). Alternatively or in combination, the device 300 can be fixed to cardiac tissue using other types of fixation mechanisms, such as, but not limited to, barbs, coils, darts, and the like.

Optionally, the device 300 can include an attachment mechanism configured to temporarily couple the device 300 to a delivery tool, e.g., for delivery and/or extraction of the device 300. In the illustrated embodiment, for example, the proximal end 310 includes a flange 318 that defines an opening. The flange 318 can be attached to a tether (e.g., by threading the tether through the opening) that extends through an elongate shaft (e.g., a catheter) to implant or extract the device 300.

In some embodiments, the device 300 is configured to sense electrical activity of the heart and/or deliver electrical stimulation to the heart via the first electrode 306a and second electrode 306b (collectively, “electrodes 306”). The first electrode 306a can serve as a cathode configured to electrically contact cardiac tissue and deliver pacing pulses thereto, and the second electrode 306b can serve as an anode and/or a return electrode. Optionally, the device 300 can be equipped with multiple cathode electrodes. Such multiple cathode electrodes can be configured to electrically contact and deliver pacing pulses to cardiac tissue of a single heart chamber, or cardiac tissue of multiple heart chambers. In some such embodiments, the multiple cathode electrodes are configured to electrically contact and deliver pacing pulses to cardiac tissue of different heart chambers. For example, one cathode electrode can be configured to electrically contact and deliver pacing pulses to atrial tissue, and another cathode electrode may be configured to electrically contact and deliver pacing pulses to ventricular tissue.

The electrodes 306 can be configured in many different ways. For example, one or both of the electrodes 306 can be discrete components that are mechanically coupled to the housing 302. As another example, one or both of the electrodes 306 can be defined by an outer portion of the housing 302 that is electrically conductive. The electrodes 306 can be electrically isolated from each other. In some embodiments, a portion of the housing 302 is covered by or formed from an insulative material to isolate the electrodes 306 from each other and/or to provide a desired size and shape for one or both of the electrodes 306. The electrodes 306 can be electrically coupled to at least some of the internal electronic components of the device 300 within the housing 302 (e.g., sensing circuitry, electrical stimulation circuitry, or both).

In the illustrated embodiment, the first electrode 306a is located at the distal end 308 of the housing 302. The first electrode 306a may be referred to as a tip electrode, and the fixation tines 304 can be configured to anchor the device 300 to cardiac tissue such that the first electrode 306a maintains contact with the cardiac tissue. In some examples, the housing 302 includes an end cap 312 at the distal end 308, and the end cap 312 includes a feedthrough assembly to electrically couple the first electrode 306a to the electronics within the housing 302, while electrically isolating the first electrode 306a from the remaining portions of the housing 302, e.g., including the second electrode 306b and/or other conductive portions of the housing 302

The second electrode 306b can be located on the housing 302 away from (e.g., proximal to) the first electrode 306a. As shown in FIG. 3, the housing 302 includes a first portion 314 and a second portion 316, with the first portion 314 being located proximal to the end cap 312, and the second portion 316 being located proximal to the first portion 314. The second portion 316 can optionally define at least part of a power source case that houses a power source (e.g., a battery) of the pacing device 300. In some embodiments, the second electrode 306b is located on the second portion 316, while in other embodiments, the second electrode 306b is located on the first portion 314.

In some embodiments, the second electrode 306b is a conductive portion of the housing 302 (e.g., an annular portion of the housing 302 that is made partially or entirely from a conductive material). Additionally or alternatively, the second electrode 306b can be a conductive material that is coated onto the material of the housing 302, or a discrete component (e.g., a ring electrode) that is coupled to the housing 302. The remaining portions of the housing 302 can include or be coated with an insulative material so that the second electrode 306b is electrically isolated from the rest of the housing 302 and/or from the first electrodes 306a.

FIG. 4 is a schematic block diagram illustrating electronic components of a pacing device 400 configured in accordance with embodiments of the present technology. Any of the electronic components shown in FIG. 4 can be incorporated into any of the embodiments of implantable devices described herein, such as the device 100 of FIG. 1, the device 200 of FIG. 2, or the device 300 of FIG. 3.

As shown in FIG. 4, the device 400 includes a plurality of electrodes 402a-402c that are electrically coupled to components within a housing 404 of the device 400. Although the device 400 is illustrated and described herein as having three electrodes 402a-402c (e.g., similar to the device 200 of FIG. 2), in other embodiments, the device 400 can be modified to include a different number of electrodes, such as two electrodes (e.g., similar to the device 300 of FIG. 3) or any other suitable number of electrodes.

At least some of the electrodes 402a-402c can be configured to contact tissue of one or more heart chambers, as described elsewhere herein. For example, as discussed above with respect to FIG. 2, the first electrode 402a can be configured to electrically contact and deliver electrical signals to tissue of a first heart chamber (e.g., ventricular tissue), and the second electrode 402b can be configured to electrically contact and deliver electrical signals to tissue of a second, different heart chamber (e.g., atrial tissue). The third electrode 402c can be an anode and/or return electrode that does not electrically contact heart tissue. Optionally, either the first electrode 402a or the second electrode 402b can be omitted, or the device 400 can include additional electrodes that electrically contact and deliver electrical signals to tissue of a heart chamber (e.g., the first heart chamber, the second heart chamber, or another heart chamber).

The device 400 includes a plurality of electronic components within the housing 404, such as switch circuitry 406, sensing circuitry 408, therapy generation circuitry 410, one or more sensors 412, processing circuitry 414, communication circuitry 416, memory 418, and/or a power source 420. The various circuitry can be or include programmable or fixed function circuitry configured to perform the operations described herein. One or more of the components of the device 400 shown in FIG. 4 can be part of an electronics assembly. For example, one or more of the switch circuitry 406, sensing circuitry 408, therapy generation circuitry 410, sensor(s) 412, processing circuitry 414, communication circuitry 416, and/or memory 418 can be mounted on a circuit board of an electronics assembly of the device 400.

The switch circuitry 406 can include one or more switches (e.g., a switch matrix, switch arrays, or other collection of switches), multiplexers, transistors, and/or other electrical circuitry. The switch circuitry 406 can selectively couple one or more of the electrodes 402a-402c to other components of the device 400 (e.g., the sensing circuitry 408 and/or the therapy generation circuitry 410). The subset of the electrodes 402a-402c to be used can depend on the particular operation of the device 400 that is being performed, such as whether the device 400 is sensing or delivering therapy, the locations of the heart being monitored or treated, etc. In some embodiments, the processing circuitry 414 determines which subset of the electrodes 402a-402c should be used for a particular operation, and controls the switch circuitry 406 to selectively couple those electrodes to the appropriate components of the device 400.

The sensing circuitry 408 can monitor signals from at least one of electrodes 402a-402c to monitor electrical activity of the heart, impedance, and/or other electrical phenomena. Sensing can be performed to determine heart rates and/or heart rate variability; and/or to detect ventricular dyssynchrony, arrhythmias (e.g., tachyarrhythmias), and/or other electrical signals. The sensing circuitry 408 can include filters, amplifiers, analog-to-digital converters, and/or other circuitry configured to sense cardiac electrical signals via one or more of the electrodes 402a-402c.

In some embodiments, the switch circuitry 406 as controlled by the processing circuitry 414 selectively couples the sensing circuitry 408 to selected combinations of the electrodes 402a-402c, e.g., to selectively sense the electrical activity of one or more chambers of the heart. For example, the switch circuitry 406 can couple each of the first electrode 402a and the second electrode 402b (in combination with the third electrode 402c) to respective sensing channels provided by the sensing circuitry 408 to sense electrical signals from the cardiac tissues in electrical contact with the first electrode 402a (e.g., ventricular tissue) and the second electrodes 402b (e.g., atrial tissue), respectively. In some embodiments, the sensing circuitry 408 is configured to detect events, (e.g., depolarizations) within the cardiac electrical signals, and to provide indications thereof to the processing circuitry 414. In this manner, the processing circuitry 414 can determine the timing of atrial and/or ventricular depolarizations, and can control the delivery of cardiac pacing (e.g., AV synchronized cardiac pacing) based thereon.

The therapy generation circuitry 410 can generate electrical stimulation signals, such as cardiac pacing pulses. The therapy generation circuitry 410 can be electrically coupled to one or more of the electrodes 402a-402c to deliver pulses to a portion of cardiac muscle within the heart via one or more of the electrodes 402a-402c. In some embodiments, the therapy generation circuitry 410 delivers pacing stimulation in the form of electrical pulses. The therapy generation circuitry 410 can include charging circuitry, and one or more charge storage devices (e.g., capacitors). Optionally, the therapy generation circuitry 410 can include switches and/or other circuitry to control when the charge storage devices are discharged to the electrodes 402a-402c.

The switch circuitry 406 as controlled by the processing circuitry 414 can direct electrical stimulation signals from the therapy generation circuitry 410 to a selected combination of the electrodes 402a-402c having selected polarities, e.g., to selectively deliver pacing pulses to the RA, RV, LV, and/or the interventricular septum of the heart. For example, in order to pace one or both of the ventricles, the switch circuitry 406 can electrically couple the first electrode 402a (e.g., which contacts wall tissue of a ventricle or the intraventricular septum) to the therapy generation circuitry 410 as a cathode, and to one or both of the second electrode 402b or the third electrode 402c to the therapy generation circuitry 410 as an anode. As another example, in order to pace the RA, the switch circuitry 406 can couple the second electrode 402b (e.g., which contacts the RA endocardium) to the therapy generation circuitry 410 as a cathode, and to one or both of the first electrode 402a or the third electrode 402c to the therapy generation circuitry 410 as an anode.

The processing circuitry 414 can include one or more processors, such as a microprocessor, a controller, a digital signal processor (DSP), an application specific integrated circuit (ASIC), a field-programmable gate array (FPGA), or equivalent discrete or analog logic circuitry. In some embodiments, the processing circuitry 414 can include multiple components, such as any combination of one or more microprocessors, controllers, DSPs, ASICs, and/or FPGAs, as well as other discrete or integrated logic circuitry. The functions attributed to the processing circuitry 414 herein may be embodied as software, firmware, hardware, or any combination thereof.

The processing circuitry 414 can control the therapy generation circuitry 410 to deliver stimulation therapy to a patient's heart according to therapy parameters, which can be stored in the memory 418. For example, the processing circuitry 414 can control the therapy generation circuitry 410 to deliver electrical pulses with the amplitudes, pulse widths, rates, frequencies, and/or electrode polarities specified by the therapy parameters. In this manner, the therapy generation circuitry 410 can deliver pacing pulses to the heart via one or more of the electrodes 402a-402c. The device 400 can use any combination of the electrodes 402a-402c to deliver therapy and/or detect electrical signals from the patient.

The memory 418 (e.g., a data storage device or other non-transitory medium) can store computer-readable instructions that, when executed by the processing circuitry 414, cause the device 400 to perform the various operations described herein. The memory 418 can include any volatile, non-volatile, magnetic, optical, or electrical media, such as a random-access memory (RAM), read only memory (ROM), non-volatile RAM (NVRAM), electrically-erasable programmable ROM (EEPROM), flash memory, or any other digital or analog media.

The sensor(s) 412 can include one or more sensing elements that transduce patient physiological activity to an electrical signal to sense values of a respective patient parameter. Sensor(s) 412 can include one or more motion sensors, optical sensors, chemical sensors, temperature sensors, pressure sensors, and/or any other types of sensors. The sensor(s) 412 can output patient parameter values to the processing circuitry 414 that can be used as feedback to control sensing and/or delivery of therapy by the device 400.

For example, the sensor(s) 412 can include at least one motion sensor, such as one or more inertial measurement units (IMUs), accelerometers, gyroscopes, electrical or magnetic field sensors, and/or other devices capable of detecting motion and/or the position of the device 400. The motion of the device 400 detected by the motion sensor may be indicative of cardiac events (e.g., paced activation of the ventricles), blood flow through the heart, patient posture, patient activity, and/or noise. The processing circuitry 414 can control and/or monitor the motion data produced by the motion sensor to identify one or more features of the cardiac contraction within the signal (e.g., on a beat-by-beat basis or otherwise) to facilitate delivery of therapy (e.g., delivery of ventricular pacing pulses in an atrial-synchronized manner). Optionally, the processing circuitry 414 can use the motion data to detect a current activity level of the patient, which can be used for rate-responsive pacing of the patient's heart.

The communication circuitry 416 is configured to allow the device 400 to wirelessly communicate with another device, such as a device external to the patient's body (e.g., the external device 110 of FIG. 1) and/or another device under the control of the processing circuitry 414. For instance, the processing circuitry 414 can receive updates to operational parameters from the other device, and/or can provide collected data, (e.g., sensed heart activity and/or other patient parameters) to the other device via the communication circuitry 416. The communication circuitry 416 can use radiofrequency (RF) communication techniques (e.g., via an antenna) and/or any other suitable communication modality.

The power source 420 delivers operating power to various components of the device 400. The power source 420 can include one or more batteries, each of which can independently be rechargeable or non-rechargeable. Recharging of the power source 420 can be accomplished through proximal inductive interaction between an external charger and an inductive charging coil within the device 400. Alternatively or in combination, recharging of the power source 420 can be accomplished using an energy harvesting mechanism 422 of the device 400. Additional details of energy harvesting mechanisms suitable for use with the embodiments herein are provided in Section II below.

The components of the device 400 illustrated in FIG. 4 can be modified in many different ways. For example, any of the components shown in FIG. 4 can be combined with each other, e.g., the switch circuitry 406 can be incorporated into the sensing circuitry 408 and/or the therapy generation circuitry 410. Any of the components shown in FIG. 4 can be divided into smaller subcomponents. Some of the components in FIG. 4 are optional and may be omitted (e.g., the switch circuitry 406 and/or sensor(s) 412). The device 400 can also include additional components not shown in FIG. 4. For example, the device 400 can include power management circuitry coupled to the power source 420 to allow the processing circuitry 414 to monitor the status of the power source 420 (e.g., charge level, charging rate, net power into and/or out of the power source 420, remaining battery life).

The components of the device 400 shown in FIG. 4 represent functionality that can be included in any of the devices of the present technology. The components illustrated in FIG. 4 can include any discrete and/or integrated electronic circuit components that implement analog and/or digital circuits capable of producing the functions attributed to the components herein. For example, the components can include analog circuits, such as amplification circuits, filtering circuits, and/or other signal conditioning circuits. The components can also include digital circuits, such as combinational or sequential logic circuits, memory devices, and the like. The functions attributed to the components of FIG. 4 may be embodied as one or more processors, hardware, firmware, software, or any combination thereof. The depiction of different features as separate blocks in FIG. 4 is intended to highlight different functional aspects, and does not necessarily imply that such components must be realized by separate hardware or software components. Rather, functionality associated with one or more components may be performed by separate hardware or software components, or integrated within common or separate hardware or software components. For example, although illustrated as separate functional components in FIG. 4, some or all of the functionality attributed to the switch circuitry 406, sensing circuitry 408, therapy generation circuitry 410, sensor(s) 412, and/or communication circuitry 416 can alternatively or additionally be implemented by the processing circuitry 414, or vice-versa.

### II. Multi-Directional Energy Harvesting Mechanisms

In some embodiments, the present technology provides implantable devices that include an energy harvesting mechanism (also known as an “energy harvester” or “harvester”). The power capacity of a power source of an implantable device may be limited due to size constraints, such as if the device is implanted within a small space within the patient's body (e.g., within a single heart chamber) and/or to avoid the device interfering with normal physiological function, as well as safety considerations. To prolong the usable life of such implantable devices, an energy harvesting mechanism can be used to generate energy in situ to recharge the power source.

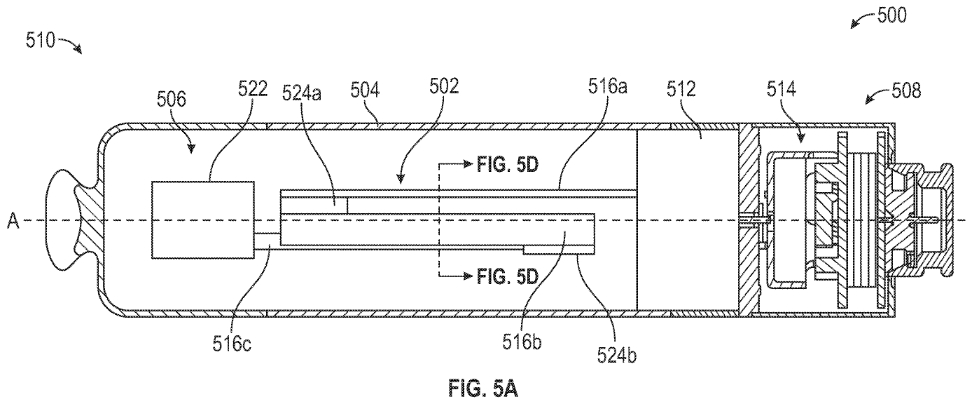

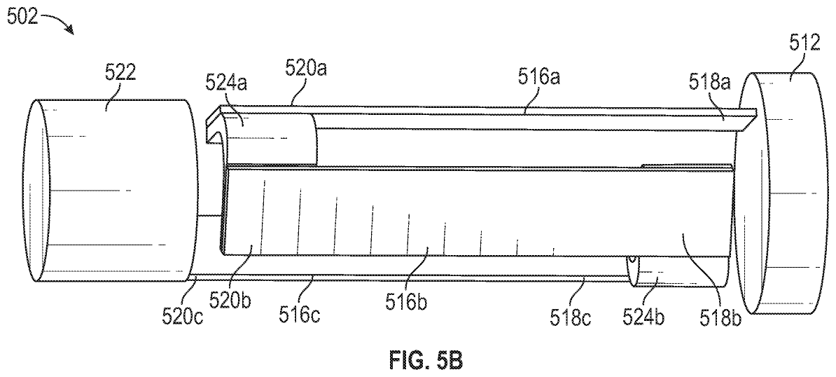

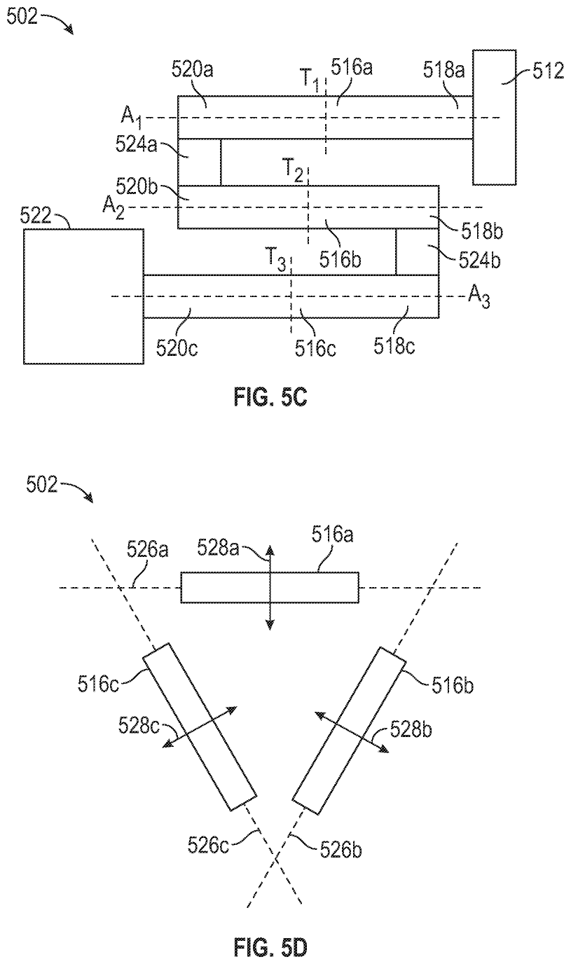

FIGS. 5A-5D illustrate a device 500 including an energy harvesting mechanism 502, in accordance with embodiments of the present technology. Specifically, FIG. 5A is a side cross-sectional view of the device 500, FIG. 5B is a perspective view of the energy harvesting mechanism 502, FIG. 5C illustrates the energy harvesting mechanism 502 in a flattened configuration, and FIG. 5D is an end cross-sectional view of the energy harvesting mechanism 502.

Referring first to FIG. 5A, the device 500 can be an implantable device, such as a pacing device configured to monitor activity of a patient's heart and provide electrical stimulation to the heart. In such embodiments, the device 500 can include any of the features of the devices described above in connection with FIGS. 1-4 (e.g., electrodes, fixation mechanism, circuitry, and/or other electronic components). In other embodiments, however, the device 500 can be a different type of implantable medical device.

The device 500 includes a housing 504 having a distal end 508 and a proximal end 510. The housing 504 can have an elongate shape having a longitudinal axis A extending from the distal end 508 to the proximal end 510. The housing 504 defines an interior cavity 506 containing the energy harvesting mechanism 502 and other components of the device 500, such as a power source 512 and an electronics assembly 514. When the device 500 is implanted in a patient's body, the energy harvesting mechanism 502 generates energy from physiological motion. For example, in some embodiments, the device 500 is configured to be implanted within a heart chamber of the patient and generates energy from cardiac motion (e.g., motion of the heart wall to which the device 500 is affixed) and/or blood flow through the heart chamber. The energy produced by the energy harvesting mechanism 502 can be used to charge the power source 512, which in turn powers the operation of the device 500 (e.g., the electronics assembly 514).

Referring next to FIGS. 5A-5D together, the energy harvesting mechanism 502 includes a plurality of piezoelectric elements 516a-516c (collectively, “piezoelectric elements 516”) that convert mechanical energy into electrical energy via the piezoelectric effect. Although FIGS. 5A-5D illustrate three piezoelectric elements 516, in other embodiments, the device 500 can include a different number of piezoelectric elements 516, as described further below. Each piezoelectric element 516 can be made partially or entirely out of a piezoelectric material, such as a piezoelectric ceramic (e.g., lead zirconate titanate (PZT)), a single crystal piezoelectric material (e.g., lead magnesium niobate-lead titanate (PMN-PT)), a piezoelectric polymer (e.g., polyvinylidene difluoride (PVDF)), or a piezoelectric composite (e.g., a piezoelectric ceramic embedded in a polymer matrix, such as a macro fiber composite). In some embodiments, some or all of the piezoelectric elements 516 are piezoelectric bimorphs (e.g., piezoelectric bimorph beams) including two active layers of a piezoelectric material (e.g., an upper layer and a lower layer). Optionally, a piezoelectric bimorph can include a passive layer made out of a non-piezoelectric material (e.g., a polymer or a metal) that serves as a support for the active layers and for electrical connectivity. In other embodiments, however, some or all of the piezoelectric elements 516 can be a piezoelectric unimorph having a single active layer.

The piezoelectric elements 516 can have any suitable geometry. In some embodiments, each piezoelectric element 516 is a flexible elongate member (e.g., a beam, plate, shaft, rod, fiber) extending from a respective first (e.g., distal) end portion to a respective second (e.g., proximal) end portion. For example, as shown in FIG. 5C, the first piezoelectric element 516a can have a distal end portion 518a and a proximal end portion 520a, with a first longitudinal axis A₁ extending between the distal end portion 518a and the proximal end portion 520a, and a first transverse axis T₁ orthogonal to the first longitudinal axis A₁. The second piezoelectric element 516b can have a distal end portion 518b and a proximal end portion 520b, with a second longitudinal axis A₂ extending between the distal end portion 518b and the proximal end portion 520b, and a second transverse axis T₂ orthogonal to the second longitudinal axis A₂. The third piezoelectric element 516c can have a distal end portion 518c and a proximal end portion 520c, with a third longitudinal axis A₃ extending between the distal end portion 518c and the proximal end portion 520c, and a third transverse axis T₃ orthogonal to the third longitudinal axis A₃. Although the piezoelectric elements 516 are each depicted as having a rectangular shape with a uniform width and thickness, in other embodiments, some or all of the piezoelectric elements 516 can have a different shape, such as a tapered shape with a varying width and/or thickness.

Referring to FIGS. 5A-5C together, the piezoelectric elements 516 can be coupled to each other in series, with the first piezoelectric element 516a being coupled to the second piezoelectric element 516b, and the second piezoelectric element 516b being coupled to the third piezoelectric element 516c. The first piezoelectric element 516a can be coupled to a fixation region within the housing 504, which can be part of the housing 504 or part of another component that is in a fixed spatial configuration relative to the housing 504. In the illustrated embodiment, for example, the first piezoelectric element 516a can be mechanically and electrically coupled to the power source 512 that is charged by the current produced by the deflection of the piezoelectric elements 516, and the power source 512 can be mounted at a fixed position within the housing 504. In other embodiments, the first piezoelectric element 516a can be mounted to an interior wall (e.g., a bulkhead) within the housing 504, and can be electrically coupled to the power source 512 via electrical interconnections (e.g., wires, traces, etc., or any suitable combination thereof) extending through or along the wall. Optionally, the fixation region can be a bearing or similar component that allows the energy harvesting mechanism 502 to passively rotate to self-align with the direction of input motion, but is mounted at a fixed axial position within the housing 504, as described in U.S. Provisional Application No. [Attorney Docket No. A0009976US01; MDTCR.008P], entitled “Energy Harvester with Rotational Alignment,” filed Apr. 17, 2023, which is incorporated by reference herein in its entirety.

The third piezoelectric element 516c can be coupled to a harvester mass 522 (also known as a “proof mass” or “inertial mass”), which can be movable within the interior cavity 506 of the housing 504, as described in greater detail below. The third piezoelectric element 516c can be coupled to the harvester mass 522 via bonding, welding, mechanical connections (e.g., interference fit, snap fit), fasteners, and/or any other suitable attachment technique.

In some embodiments, the piezoelectric elements 516 are coupled to each other in an end-to-end, folded configuration (e.g., a fan-folded, zig-zag, or serpentine configuration). For example, the distal end portion 518a of the first piezoelectric element 516a can be affixed relative to the housing 504 (e.g., via a direct connection to the housing 504 or indirectly via another component, such as the power source 512 or an interior wall within the housing 504). The proximal end portion 520a of the first piezoelectric element 516a can be coupled to the proximal end portion 520b of the second piezoelectric element 516b (e.g., via a first joint 524a). The distal end portion 518b of the second piezoelectric element 516b can be coupled to the distal end portion 518c of the third piezoelectric element 516c (e.g., via a second joint 524b). The proximal end portion 520c of the third piezoelectric element 516c can be coupled to the harvester mass 522. The piezoelectric elements 516 can be arranged so that their respective longitudinal axes A₁, A₂, A₃ are aligned with (e.g., parallel to or within 10°, 5°, 2°, or 1° of being parallel to) each other and/or to the longitudinal axis A of the housing 504.

The joints 524a, 524b can be blocks, strips, plates, brackets, wires, interposers, etc., that mechanically couple the respective pairs of piezoelectric elements 516 to each other. The joints 524a, 524b can be sufficiently rigid to maintain the piezoelectric elements 516 in the desired spatial arrangement relative to each other, and/or to reduce excessive flexing of the joints 524a, 524b that dissipates mechanical energy. Optionally, the joints 524a, 524b can have some degree of flexibility to provide an elastic rebound effect that dynamically amplifies the deflection of the piezoelectric elements 516 in response to a motion input.

In some embodiments, the joints 524a, 524b include electrically conductive materials (e.g., metals, conductive polymers) to electrically couple the piezoelectric elements 516 to each other. Accordingly, current produced by the second piezoelectric element 516b and the third piezoelectric element 516c can be transmitted to the power source 512 via the joints 524a, 524b. For example, the joints 524a, 524b can be made entirely out of a metal (e.g., brass, copper), and can be attached to the corresponding piezoelectric elements 518 via soldering, brazing, conductive adhesives, etc. As another example, the joints 524a, 524b can be made out of a combination of an insulative material (e.g., polyimide, polycrystalline ceramic such as alumina) and a conductive material (e.g., conductive traces, fine wires) using techniques such as molding and hot isostatic processing and wire bonding. In other embodiments, however, one or both of the joints 524a, 524b can be omitted, and the piezoelectric elements 516 can instead be directly connected to each other (e.g., via welding, adhesives, fasteners) or integrally formed with each other.

The configuration of the piezoelectric elements 516 described herein can decrease the overall resonant frequency of the energy harvesting mechanism 502 to a range suitable for efficient harvesting from physiological motion, while also reducing the spatial footprint of the energy harvesting mechanism 502. For example, the resonant frequency of the energy harvesting mechanism 502 can be within a range from 1 Hz to 50 Hz, 1 Hz to 30 Hz, 1 Hz to 20 Hz, 1 Hz to 10 Hz, 5 Hz to 10 Hz, 5 Hz to 15 Hz, 10 Hz to 20 Hz, 10 Hz to 15 Hz, 10 Hz to 30 Hz, 15 Hz to 20 Hz, 15 Hz to 25 Hz, 20 Hz to 30 Hz, 20 Hz to 25 Hz, or 25 Hz to 30 Hz. The length of the energy harvesting mechanism 502 (excluding the harvester mass 522) can be less than or equal to 30 mm, 25 mm, 20 mm, or 15 mm; and/or within a range from 10 mm to 30 mm, 10 mm to 20 mm, 14 mm to 18 mm, or 15 mm to 25 mm. The maximum cross-sectional dimension of the energy harvesting mechanism 502 (e.g., maximum width and/or diameter measured orthogonal to the longitudinal axis A) (excluding the harvester mass 522) can be less than or equal to within 15 mm, 12 mm, 10 mm, or 8 mm; and/or within a range from 5 mm to 15 mm, or 5 mm to 10 mm.

When the device 500 is subjected to external forces from physiological motion, the inertia of the harvester mass 522 can cause deflection of the proximal end portion 520c of the third piezoelectric element 516c. The forces can be transmitted to the second piezoelectric element 516b and the first piezoelectric element 516a by virtue of the interconnections between these components. Accordingly, some or all of the piezoelectric elements 516 can be elastically deformed relative to the housing 504 and to the fixed distal end portion 518a of the first piezoelectric element 516a. For example, some or all of the piezoelectric elements 516 can be deformed from a resting, straightened configuration (shown in FIG. 5A) to a bent configuration. The resulting mechanical strain in the piezoelectric element(s) 516 can produce an electrical current that can be used to charge the power source 512.

In some embodiments, each piezoelectric element 516 is characterized by a principal bending direction (also referred to interchangeably as a “preferred bending direction,” “preferred flexing direction,” or “preferred motion axis”). The principal bending direction of a piezoelectric element 516 can correspond to the direction of input motion that maximizes the power output of the piezoelectric element 516 and/or minimizes the amount of force to deflect the piezoelectric element 516. When the direction of input motion is offset from the principal bending direction, the power output of the piezoelectric element 516 can be reduced and/or more force may be required to deflect the piezoelectric element 516. In some instances, the power output can be minimal when the direction of input motion is orthogonal to the principal bending direction. Accordingly, it can be advantageous to arrange the piezoelectric elements 516 to have different principal bending directions so that regardless of the direction of input motion, at least one piezoelectric element 516 is sufficiently deflected to provide at least a minimum level of power output. In some embodiments, for any arbitrary direction of input motion, the energy harvesting mechanism 502 produces a power output (e.g., a minimum and/or average power output) of at least 1 μW, 1.5 μW, 2 μW, 2.5 μW, 3 μW, 3.5 μW, 4 μW, 4.5 μW, 5 μW, 5.5 μW, 6 μW, 6.5 μW, 7μ, 7.5μ, 8μ, 8.5μ, 9μ, 9.5μ, or 10μ W.

The principal bending direction of a piezoelectric element 516 can depend on the geometry of the piezoelectric element 516. For example, as shown in FIG. 5D, the piezoelectric elements 516 can each have a flattened body (e.g., a beam, strip, film, plate) that defines a respective plane, e.g., the first piezoelectric element 516a defines a first plane 526a that is aligned with (e.g., parallel to) the first transverse axis T₁ and the first longitudinal axis A₁, the second piezoelectric element 516b defines a second plane 526b that is aligned with (e.g., parallel to) the second transverse axis T₂ and the second longitudinal axis A₂, and the third piezoelectric element 516c defines a third plane 526c that is aligned with (e.g., parallel to) the third transverse axis T₃ and the third longitudinal axis A₃. Accordingly, the principal bending direction of the first piezoelectric element 516a can be a first direction 528a that is orthogonal to the first plane 526a, the principal bending direction of the second piezoelectric element 516b can be a second direction 528b that is orthogonal to the second plane 526b, and the principal bending direction of the third piezoelectric element 516c can be a third direction 528c that is orthogonal to the third plane 526c.

In some embodiments, the piezoelectric elements 516 face different directions, such that the principal bending directions of the piezoelectric elements 516 differ from each other, thus allowing for efficient energy harvesting from different directions of input motion. For instance, the piezoelectric elements 516 can have different orientations so that the first plane 526a, second plane 526b, and third plane 526c are offset from each other, such as by an angle greater than or equal to 60°, 70°, 80°, 90°, 100°, 110°, 120°, 130°, 140°, 150, 160°, or 170°. Accordingly, the first direction 528a, second direction 528b, and third direction 528c are also offset from each other, such as by an angle greater than or equal to 5°, 10°, 20°, 30°, 40°, 50°, 60°, 70°, 80°, 90°, 100°, 110°, or 120°. In the illustrated embodiment, the piezoelectric elements 516 are radially arranged around the longitudinal axis A of the housing 504 in a radially symmetric configuration, such that the angle between neighboring planes is 60° and the principal bending directions are offset by 120°. In other embodiments, however, the piezoelectric elements 516 can be arranged differently, such as in a non-radially symmetric configuration, an overlapping configuration, etc.

Referring again to FIG. 5A, the power source 512 (shown schematically) can be or include one or more accumulators (e.g., rechargeable batteries) that are electrically coupled to the energy harvesting mechanism 502 to store the energy produced by the energy harvesting mechanism 502. In the illustrated embodiment, the power source 512 is located distal to the energy harvesting mechanism 502, and is interposed between the energy harvesting mechanism 502 and the electronics assembly 514 at the distal end 508 of the housing 504. In other embodiments, however, the power source 512 can have a different shape and/or can be located at a different portion within the housing 504. For example, the power source 512 can be a tubular structure that surrounds at least a portion of the energy harvesting mechanism 502, such as a distal portion of the energy harvesting mechanism 502. In such embodiments, the tubular power source can include a lumen extending therethrough, such that at least a portion of the energy harvesting mechanism 502 is received within the lumen. This configuration can be advantageous for reducing the overall size of the device 500 while maintaining sufficient space within the interior cavity 506 to allow for movement of the harvester mass 522 and piezoelectric elements 516.

Optionally, the device 500 can include power conditioning circuitry (not shown) electrically coupled to and interposed between the energy harvesting mechanism 502 and the power source 512. The power conditioning circuitry can be configured to perform operations such as rectification, filtering, voltage regulation, etc., of the electrical signal produced by the energy harvesting mechanism 502, before transmission to the power source 512.

The power source 512 is electrically coupled to the electronics assembly 514 to power the operation thereof. The electronics assembly 514 can include the electronic components of the device 500, such as any of the components described above with respect to FIG. 4 (e.g., switch circuitry 406, sensing circuitry 408, therapy generation circuitry 410, sensors 412 processing circuitry 414, communication circuitry 416, and/or memory 418).

Optionally, the electronics assembly 514 can include components (e.g., processing circuitry 414 and/or other circuitry) that perform power management functions, such as monitoring the status of the power source 512 (e.g., the charge level of the power source 512; whether the charge level is increasing, decreasing, or constant; the net current and/or power into the power source 512) and/or monitoring the power output of the energy harvesting mechanism 502 (e.g., amount of current and/or power produced by the energy harvesting mechanism 502), power consumption of the electronics assembly 514, etc. In some embodiments, the polarity of each piezoelectric element 516 (e.g., the polarity of each active layer of each piezoelectric element 516, when piezoelectric bimorphs are used) varies depending on the orientation of the piezoelectric element 516 relative to the direction of input motion. Accordingly, the electronics assembly 514 can include circuitry that monitors and independently adjusts the polarity of each piezoelectric element 516 to ensure the correct phase relationship between piezoelectric elements 516. For instance, such circuitry can include a full-bridge rectifier using low-forward voltage diodes (e.g., Schottky diodes), synchronous rectifiers based on MOSFET switches and/or other passive components, and/or other suitable approaches known to those of skill in the art.

The configuration of the device 500 of FIGS. 5A-5D can be modified in many different ways. For example, although the piezoelectric elements 516 are depicted as being connected in series, in other embodiments, the piezoelectric elements 516 can be connected in parallel, or each piezoelectric element 516 can be routed individually to the power source 512 to allow for selective enabling and disabling of active and inactive elements, respectively. Additionally, although the piezoelectric elements 516 are illustrated as being discrete components connected to each other via the joints 524a, 524b, in other embodiments, the piezoelectric elements 516 can instead be a single monolithic component that is bent or otherwise formed into the folded configuration shown in FIGS. 5A-5D, such as an elongate metal strip with upper and lower piezoelectric layers. This approach can improve the reliability of the energy harvesting mechanism 502 by obviating the need for joints, bonding, welding, adhesives, etc., to connect the piezoelectric elements 516 to each other.

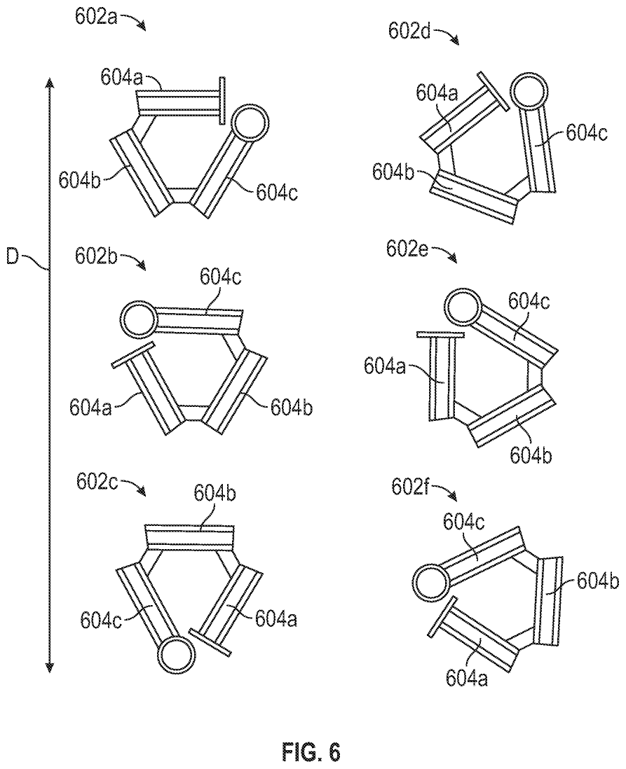

FIG. 6 is a partially schematic end view of an energy harvesting mechanism in a plurality of different orientations 602a-602f, in accordance with embodiments of the present technology. The energy harvesting mechanism included three piezoelectric bimorph beams 604a-604c connected in series, similar to the configuration of energy harvesting mechanism 502 of FIGS. 5A-5D. The first beam 604a included a first end coupled to a vertical shake table, and a second end coupled to the second beam 604b via a first joint; the second beam 604b included a first end coupled to the first beam 604a via the first joint, and a second end coupled to the third beam 604c via a second joint; and the third beam 604c included a first end coupled to the second beam 604b via the second joint and a second end coupled to a tip mass. The beams 604a-604c were arranged in a triangular configuration with each beam offset from the neighboring beams by 60°.

The energy harvesting mechanism was excited with mechanical input motion in the form of a 20 Hz sine wave along movement direction D, and the power output was measured for the six orientations 602a-602f shown in FIG. 6. In orientations 602a-602c, the first beam 604a, the third beam 604c, and the second beam 604b were oriented perpendicular to the movement direction D, respectively. In orientations 602d-602f, the first beam 604a, the second beam 604b, and the third beam 604c were at a 45° offset from the movement direction D, respectively.

As shown in Table 1 below, the energy harvesting mechanism produced power at each orientation. The highest power output was observed with orientation 602a, in which the first beam 604a was perpendicular to the movement direction D, such that the movement direction D was parallel to the principal bending direction of the first beam 604a. The lowest power output was observed with orientation 602e, in which the first beam 604a was parallel to the movement direction D, such that the movement direction D was perpendicular to the principal bending direction of the first beam 604a.

TABLE 1
Test Results for Energy Harvesting
Mechanism in Different Orientations

| | Average Power |
|---|---|
| Orientation | Output (μW) |
| 602a | 8 |
| 602b | 2.5 |
| 602c | 4.5 |
| 602d | 3 |
| 602e | 1.5 |
| 602f | 4 |

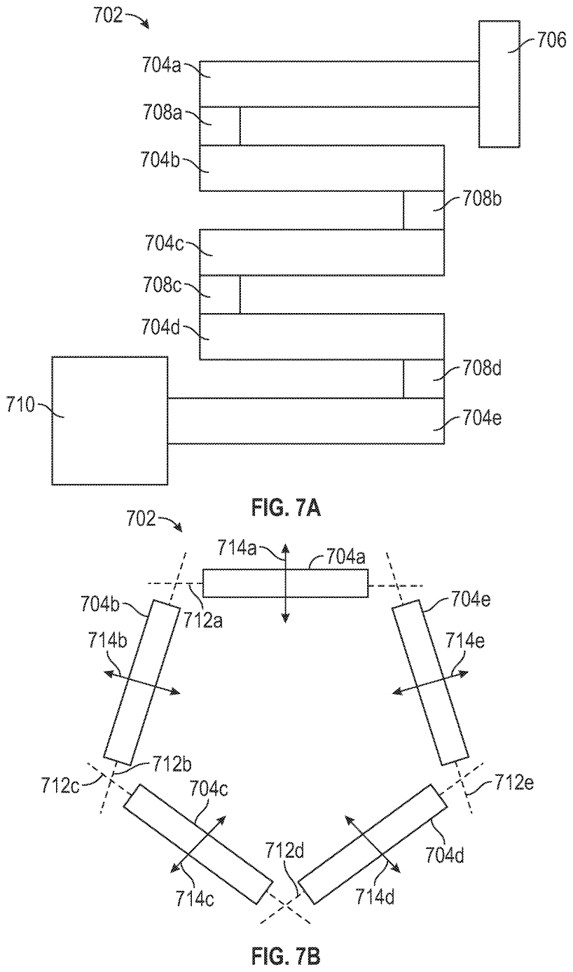

FIGS. 7A and 7B are partially schematic illustrations of another energy harvesting mechanism 702 configured in accordance with embodiments of the present technology. Specifically, FIG. 7A illustrates the energy harvesting mechanism 702 in a flattened configuration, and FIG. 7B is an end cross-sectional view of the energy harvesting mechanism 702. The energy harvesting mechanism 702 can be incorporated into any of the devices described herein (e.g., the devices of FIGS. 1-5D). The energy harvesting mechanism 702 can be generally similar to the energy harvesting mechanism 502 of FIGS. 5A-5D, except that the energy harvesting mechanism 702 includes five piezoelectric elements 704a-704e (collectively, “piezoelectric elements 704”). Accordingly, the following discussion of the energy harvesting mechanism 702 will be limited to those features that differ from the embodiments of FIGS. 5A-5D.

Referring first to FIG. 7A, the piezoelectric elements 704 can be coupled to each other in series, such that the first piezoelectric element 704a is coupled to a fixation region 706 of the housing containing the energy harvesting mechanism 502, and to the second piezoelectric element 704b (e.g., via a first joint 708a); the second piezoelectric element 704b is coupled to the third piezoelectric element 706c (e.g., via a second joint 708b); the third piezoelectric element 706c is coupled to the fourth piezoelectric element 704d (e.g., via a third joint 708c); the fourth piezoelectric element 704d is coupled to the fifth piezoelectric element 704e (e.g., via a fourth joint 708d); and the fifth piezoelectric element 704e is coupled to a harvester mass 710. The joints 708a-708d can mechanically and/or electrically interconnect neighboring pairs of piezoelectric elements 704, and can be identical or generally similar to the joints 524a, 524b of FIGS. 5A-5D. Alternatively, the piezoelectric elements 704a-704e can be connected in parallel, or each piezoelectric element can be routed individually to the power source to allow for selective enabling and disabling of active and inactive elements, respectively. Moreover, the piezoelectric elements 704a-704e can alternatively be provided as a single monolithic component that is bent or otherwise formed into the folded configuration shown in FIGS. 7A and 7B.

The piezoelectric elements 704 can be arranged with their respective longitudinal axes aligned with (e.g., parallel to or within 10°, 5°, 2°, or 1° of being parallel to) each other and/or to the longitudinal axis of the housing containing the energy harvesting mechanism 702. When the piezoelectric elements 704 are in an assembled configuration (FIG. 7B), the piezoelectric elements 704 can each face a different direction, so that the respective planes 712a-712e defined by the corresponding longitudinal and transverse axes of each piezoelectric element 704 are offset from each other, and/or the respective principal bending directions 714a-712e of the piezoelectric elements 704 are offset from each other. In the illustrated embodiment, the piezoelectric elements 704 are arranged in a radially symmetric configuration (e.g., a pentagonal configuration), such that the angle between neighboring planes is 108° and the principal bending directions are offset by 72°. In other embodiments, however, the piezoelectric elements 704 can be arranged differently, such as in a non-radially symmetric configuration, an overlapping configuration, etc.

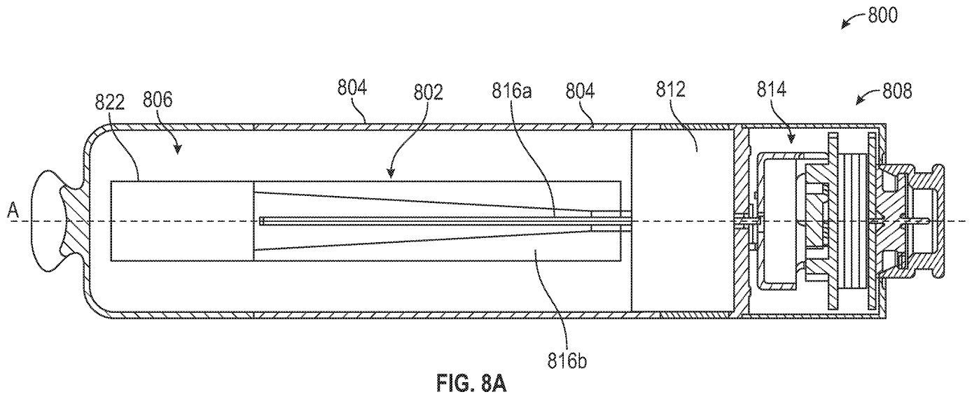

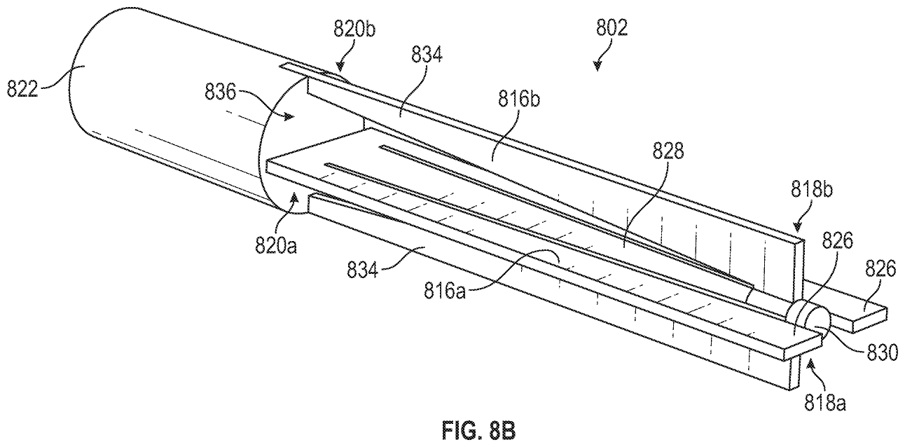

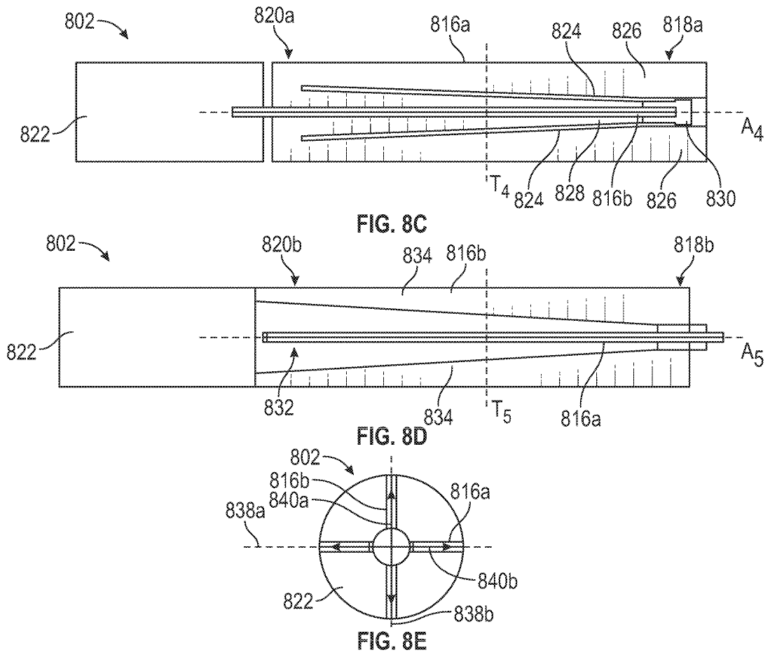

FIGS. 8A-8E illustrate a device 800 including an energy harvesting mechanism 802, in accordance with embodiments of the present technology. Specifically, FIG. 8A is a side cross-sectional view of the device 800, FIG. 8B is a perspective view of the energy harvesting mechanism 802, FIG. 8C is a top view of the energy harvesting mechanism 802, FIG. 8D is a side view of the energy harvesting mechanism 802, and FIG. 8E is an end view of the energy harvesting mechanism 802.

Referring first to FIG. 8A, the device 800 can be an implantable device, such as a pacing device configured to monitor activity of a patient's heart and provide electrical stimulation to the heart. In such embodiments, the device 800 can include any of the features of the devices described above in connection with FIGS. 1-4 (e.g., electrodes, fixation mechanism, circuitry, and/or other electronic components). In other embodiments, however, the device 800 can be a different type of implantable medical device.

The device 800 can be generally similar to the device 500 of FIGS. 5A-5D. For example, the device 800 can include a housing 804 defining an interior cavity 806 that contains the energy harvesting mechanism 802, a power source 812, and an electronics assembly 814. Accordingly, like numbers (e.g., housing 804 versus housing 504) are used to identify similar or identical components, and the following discussion of the device 800 will be limited to those features that differ from the embodiments described with respect to FIGS. 5A-5D.

Referring to FIGS. 8A and 8B together, the energy harvesting mechanism 802 includes a pair of piezoelectric elements 816a, 816b (collectively, “piezoelectric elements 816”). Each piezoelectric element 816 can be made partially or entirely out of a piezoelectric material, such as a piezoelectric ceramic (e.g., PZT), a single crystal piezoelectric material (e.g., PMN-PT), a piezoelectric polymer (e.g., PVDF), or a piezoelectric composite (e.g., a piezoelectric ceramic embedded in a polymer matrix, such as a macro fiber composite). In some embodiments, some or all of the piezoelectric elements 816 are piezoelectric bimorphs (e.g., piezoelectric bimorph beams) including two active layers of a piezoelectric material (e.g., an upper layer and a lower layer). Optionally, a piezoelectric bimorph can include a passive layer made out of a non-piezoelectric material (e.g., a polymer or a metal) that serves as a support for the active layers. In other embodiments, however, some or all of the piezoelectric elements 816 can be a piezoelectric unimorph having a single active layer.

The piezoelectric elements 816 can have any suitable geometry. In some embodiments, each piezoelectric element 816 is a flexible elongate member (e.g., a beam, plate, shaft, rod, fiber) extending from a respective first (e.g., distal) end portion to a respective second (e.g., proximal) end portion. For example, as shown in FIGS. 8B and 8C, the first piezoelectric element 816a can have a distal end portion 818a and a proximal end portion 820a, with a first longitudinal axis A₄ extending between the distal end portion 818a and the proximal end portion 820a, and a first transverse axis T₄ orthogonal to the first longitudinal axis A₄. As shown in FIGS. 8B and 8D, the second piezoelectric element 816b can have a distal end portion 818b and a proximal end portion 820b, with a second longitudinal axis A₅ extending between the distal end portion 818b and the proximal end portion 820b, and a second transverse axis T₅ orthogonal to the second longitudinal axis A₅.

Referring again to FIG. 8A, the first piezoelectric element 816a can be coupled to a fixation region within the housing 804. The fixation region can be part of the housing 804 or part of another component that is in a fixed spatial configuration relative to the housing 804. In the illustrated embodiment, for example, the first piezoelectric element 816a can be mechanically and electrically coupled to the power source 812 that is charged by the current produced by the deflection of the piezoelectric elements 816, and the power source 812 can be mounted at a fixed position within the housing 804. In other embodiments, the first piezoelectric element 816a can be mounted to an interior wall (e.g., a bulkhead) within the housing 804, and can be electrically coupled to the power source 812 via electrical interconnections (e.g., wires) extending through or along the wall. The second piezoelectric element 816b can be coupled to the first piezoelectric element 816a and to a harvester mass 822, which can be movable within the interior cavity 806 of the housing 804, as described in greater detail below.

Referring to FIGS. 8A-8D together, the piezoelectric elements 816 can be coupled to each other in an end-to-end, overlapping (e.g., interleaved and/or coaxial) configuration. For example, the distal end portion 818a of the first piezoelectric element 816a can be coupled to the fixation region within the housing 804 and to the second piezoelectric element 816b. The proximal end portion 802a of the first piezoelectric element 816a can be positioned near but spaced apart from the harvester mass 822. The distal end portion 818b of the second piezoelectric element 816b can be coupled to the first piezoelectric element 816a, and can be positioned near but spaced apart from the fixation region. The proximal end portion 820b of the second piezoelectric element 816b can be coupled to the harvester mass 822. The piezoelectric elements 816 can be arranged so that their respective longitudinal axes A₄, A₅ are aligned with (e.g., parallel to or within 10°, 5°, 2°, or 1° of being parallel to) each other and/or to the longitudinal axis A of the housing 804.

In some embodiments, the distal end portion 818a of the first piezoelectric element 816a includes an outer structure coupled to the fixation region, and an inner structure coupled to the second piezoelectric element 816b. As best seen in FIG. 8B and 8C, the first piezoelectric element 816a can include a pair of elongate slots 824 extending from the distal end portion 818a toward the proximal end portion 820a. The slots 824 can terminate before the proximal end portion 820a, thus defining a pair of outer members 826 (e.g., outer flaps or projections), and an inner member 828 (e.g., an inner flap or projection) between the outer members 826. The distal ends of the outer members 826 can be fixedly coupled to the housing 804 (e.g., via a direct connection or indirectly via another component), while the distal end of the inner member 828 can be coupled to the distal end portion 818b of the second piezoelectric element 816b (e.g., via a joint 830). The outer members 826 and inner member 828 can be movable relative each other (e.g., in a direction orthogonal to the longitudinal axis A₄ and the transverse axis T₄) such that these components behave like two discrete piezoelectric elements connected in series at the proximal end portion 820a, as discussed further below. In the illustrated embodiment, the slots 824 are angled away from the first longitudinal axis A₄ so that the inner member 828 is tapered with a narrower distal end and a wider proximal end, and the outer members 826 are tapered with wider distal ends and narrower proximal ends. In other embodiments, however, the geometry of the slots 824, outer members 826, and inner member 828 can be varied as desired.

As best seen in FIGS. 8B and 8D, the second piezoelectric element 816b can include an elongate opening 832 extending from the proximal end portion 820b toward the distal end portion 818b. The opening 832 can define a pair of outer members 834 (e.g., flaps, projections). In some embodiments, the opening 832 terminates before the distal end portion 818b of the second piezoelectric element 816b, such that the distal ends of the outer members 834 are connected to each other. Alternatively, the opening 832 can extend to the distal end portion 818b, such that there is a gap between the distal ends of the outer members 834. In such embodiments, the joint 830 and/or the inner member 828 of the first piezoelectric element 816a can bridge the gap between the distal ends of the outer members 834 to couple the outer members 834 to each other. In the illustrated embodiment, the opening 832 has a tapered shape with a wider proximal end and a narrower distal end, such that the outer members 834 are tapered with wider distal ends and narrower proximal ends. In other embodiments, however, the geometry of the opening 832 and outer members 834 can be varied as desired.

As shown in FIG. 8B, the first piezoelectric element 816a can be partially received within the opening 832 of the second piezoelectric element 816b, such that at least a portion of the first piezoelectric element 816a (e.g., the inner member 828) is positioned between the outer members 834 of the second piezoelectric element 816b. The distal ends of the outer members 834 of the second piezoelectric element 816b can be coupled to the distal end of the inner member 828 of the first piezoelectric element 816a (e.g., via the joint 830). The proximal ends of the outer members 834 of the second piezoelectric element 816b can be coupled to the harvester mass 822. Accordingly, the outer members 826 of the first piezoelectric element 816a, the inner member 828 of the first piezoelectric element 816a, and the outer members 834 of the second piezoelectric element 816b can effectively behave as a fan-folded assembly having three “beams” connected in series. Specifically, the outer members 826 of the first piezoelectric element 816a can collectively serve as the first “beam” with a fixed distal end, the inner member 828 of the first piezoelectric element 816a can serve as the second “beam,” and the outer members 834 of the second piezoelectric element 816b can collectively serve as the third “beam” with a proximal end coupled to the harvester mass 822.

The joint 830 can be a block, cap, strip, plate, bracket, wire, interposer, etc., that mechanically couples the first piezoelectric element 816a and the second piezoelectric element 816b to each other. The joint 830 can be generally similar to those of the joints 524a, 524b of FIGS. 5A-5D, and can include any of the features described in connection with FIGS. 5A-5D. For example, the joint 830 can include electrically conductive materials (e.g., metals, conductive polymers) to electrically couple the first piezoelectric element 816a to the second piezoelectric element 816b. In other embodiments, however, the joint 830 can be omitted, and the first piezoelectric element 816a and the second piezoelectric element 816b can instead be directly connected to each other (e.g., via welding, adhesives, fasteners) or integrally formed with each other.

The configuration of the piezoelectric elements 816 described herein can decrease the overall resonant frequency of the energy harvesting mechanism 802 to a range suitable for efficient harvesting from physiological motion, while also reducing the spatial footprint of the energy harvesting mechanism 802. For example, the resonant frequency of the energy harvesting mechanism 802 can be within a range from 1 Hz to 50 Hz, 1 Hz to 30 Hz, 1 Hz to 20 Hz, 1 Hz to 10 Hz, 5 Hz to 10 Hz, 5 Hz to 15 Hz, 10 Hz to 20 Hz, 10 Hz to 15 Hz, 10 Hz to 30 Hz, 15 Hz to 20 Hz, 15 Hz to 25 Hz, 20 Hz to 30 Hz, 20 Hz to 25 Hz, or 25 Hz to 30 Hz. The length of the energy harvesting mechanism 802 (excluding the harvester mass 822) can be less than or equal to 30 mm, 25 mm, 20 mm, or 15 mm; and/or within a range from 10 mm to 30 mm, 10 mm to 20 mm, 14 mm to 18 mm, or 15 mm to 25 mm. The maximum cross-sectional dimension of the energy harvesting mechanism 802 (e.g., maximum width and/or diameter measured orthogonal to the longitudinal axis A) (excluding the harvester mass 822) can be less than or equal to within 15 mm, 12 mm, 10 mm, or 8 mm; and/or within a range from 5 mm to 15 mm, or 5 mm to 10 mm.

When the device 800 is subjected to external forces from physiological motion, the inertia of the harvester mass 822 can cause deflection of the proximal end portion 820b of the second piezoelectric element 816b. The forces can be transmitted to the first piezoelectric element 816a by virtue of the interconnections between these components. Accordingly, one or both of the piezoelectric elements 816 can be elastically deformed relative to the housing 804 and to the fixed distal end portion 818a (e.g., the distal ends of the outer members 826) of the first piezoelectric element 816a. For example, one or both of the piezoelectric elements 816 can be deformed from a resting, straightened configuration (shown in FIG. 8A) to a bent configuration. The resulting mechanical strain in the piezoelectric element(s) 816 can produce an electrical current that can be used to charge the power source 812.

In some embodiments, each piezoelectric element 816 is characterized by a principal bending direction. As described herein, the principal bending direction of a piezoelectric element 816 can correspond to the direction of input motion that maximizes the power output of the piezoelectric element 816 and/or minimizes the amount of force to deflect the piezoelectric element 816. The piezoelectric elements 816 can be arranged to have different principal bending directions so that regardless of the direction of input motion, at least one piezoelectric element 816 is sufficiently deflected to provide at least a minimum level of power output. In some embodiments, for any arbitrary direction of input motion, the energy harvesting mechanism 802 produces a power output (e.g., a minimum and/or average power output) of at least 1 μW, 1.5 μW, 2 μW, 2.5 μW, 3 μW, 3.5 μW, 4 μW, 4.5 μW, 5 μW, 5.5 μW, 6 μW, 6.5 μW, 7 μW, 7.5 μW, 8 μW, 8.5 μW, 9 μW, 9.5 μW, or 10 μW.

The principal bending direction of a piezoelectric element 816 can depend on the geometry of the piezoelectric element 816. For example, as shown in FIG. 8E, the piezoelectric elements 816 can each have a generally flattened body (e.g., a beam, strip, film, plate) that defines a respective plane, e.g., the first piezoelectric element 816a defines a first plane 838a that is aligned with (e.g., parallel to) the first transverse axis T₄ and the first longitudinal axis A₄, and the second piezoelectric element 816b defines a second plane 838b that is aligned with (e.g., parallel to) the second transverse axis T₅ and the second longitudinal axis A₅. Due to the overlapping configuration of the first piezoelectric element 816a and the second piezoelectric element 816b, the first plane 838a can intersect the second plane 838b. The principal bending direction of the first piezoelectric element 816a can be a first direction 840a that is orthogonal to the first plane 838a, and the principal bending direction of the second piezoelectric element 816b can be a second direction 840b that is orthogonal to the second plane 838b.

In some embodiments, the piezoelectric elements 816 face different directions, such that the principal bending directions of the piezoelectric elements 816 differ from each other, thus allowing for efficient energy harvesting from different directions of input motion. For instance, the piezoelectric elements 816 can have different orientations so that the first plane 838a is offset from the second plane 838b, such as by an angle greater than or equal to 5°, 10°, 20°, 30°, 40°, 50°, 60°, 70°, 80°, 90°, 100°, 110°, 120° 130°, 140°, 150, 160°, or 170°. Accordingly, the first direction 840a can also be offset from the second direction 840b, such as by an angle greater than or equal to 5°, 10°, 20°, 30°, 40°, 50°, 60°, 70°, 80°, 90°, 100°, 110°, 120° 130°, 140°, 150, 160°, or 170°. In the illustrated embodiment, the piezoelectric elements 816 are arranged in a radially symmetric configuration, such that the angle between the planes 838a, 838b and the principal bending directions 840a, 840b is 90°. In other embodiments, however, the piezoelectric elements 816 can be arranged differently, such as in a non-radially symmetric configuration, a non-overlapping configuration, etc.

The configuration of the device 800 of FIGS. 8A-8E can be modified in many different ways. For example, although the piezoelectric elements 816 are illustrated as being discrete components connected to each other via the joint 830, in other embodiments, the piezoelectric elements 816 can instead be a single monolithic component that is bent or otherwise formed into the configuration shown in FIGS. 8A-8E, such as a single elongate metal strip with upper and lower piezoelectric layers.

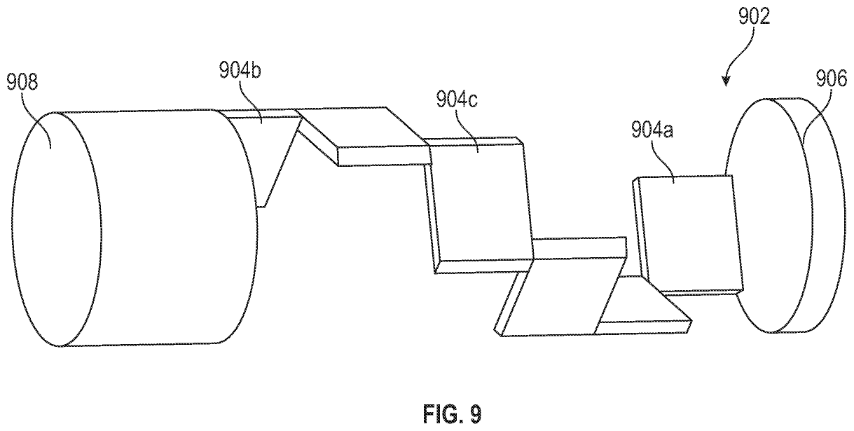

FIG. 9 is a perspective view of an energy harvesting mechanism 902 configured in accordance with embodiments of the present technology. The energy harvesting mechanism 902 can be incorporated into any of the devices described herein (e.g., the devices of FIGS. 1-5D). The energy harvesting mechanism 902 includes a plurality of piezoelectric elements coupled to each other in series, including a first piezoelectric element 904a coupled to a fixation region 906 within the housing containing the energy harvesting mechanism 902, a second piezoelectric element 904b coupled to a harvester mass 908, and one or more third piezoelectric elements 904c connected in series between the first piezoelectric element 904a and the second piezoelectric element 904b. Although FIG. 9 illustrates a total of six piezoelectric elements 904a-904c (collectively, “piezoelectric elements 904”), in other embodiments, the energy harvesting mechanism 902 can include a different number of piezoelectric elements 904, such as two, three, four, five, or more piezoelectric elements 904.

Each piezoelectric element 904 can be made partially or entirely out of a piezoelectric material, such as a piezoelectric ceramic (e.g., PZT), a single crystal piezoelectric material (e.g., PMN-PT), a piezoelectric polymer (e.g., PVDF), or a piezoelectric composite (e.g., a piezoelectric ceramic embedded in a polymer matrix, such as a macro fiber composite). In some embodiments, some or all of the piezoelectric elements 904 are piezoelectric bimorphs, while in other embodiments, some or all of the piezoelectric elements 904 are piezoelectric unimorphs. The piezoelectric elements 904 can each have any suitable geometry, such as a beam, plate, shaft, rod, fiber, or other elongate member.

As shown in FIG. 9, the piezoelectric elements 904 can be mechanically and/or electrically coupled to each other in an end-to-end and/or unfolded configuration, such that the distal end portion of each piezoelectric element 904 is coupled to the proximal end portion of the neighboring piezoelectric element 904. Although the piezoelectric elements 904 are illustrated as being directly connected to each other, in other embodiments, some or all of the piezoelectric elements 904 can instead be coupled to each other via joints. The joints can optionally provide electrical interconnectivity between neighboring piezoelectric elements 904, as described herein.

The piezoelectric elements 904 can be arranged with their respective longitudinal axes aligned with (e.g., parallel to or within 10°, 5°, 2°, or 1° of being parallel to) each other and/or to the longitudinal axis of the housing containing the energy harvesting mechanism 902. In the illustrated embodiment, the piezoelectric elements 904 are arranged in a helical configuration along the longitudinal axis of the energy harvesting mechanism 902. Each piezoelectric element 904 can be longitudinally and circumferentially offset from the neighboring piezoelectric elements 904, such that the distal end surface of each piezoelectric element 904 only partially overlaps or does not overlap the proximal end surface of the neighboring piezoelectric element 904. The piezoelectric elements 904 can each face a different direction, such that the respective planes of the piezoelectric elements 904 face different directions and each piezoelectric element 904 has a different respective principal bending direction. For instance, as shown in FIG. 9, each piezoelectric element 904 can face the central longitudinal axis of the helix, with the plane of each piezoelectric element 904 being angled relative to the planes of the neighboring piezoelectric elements 904.

Although the embodiments of FIGS. 5A-9 illustrate energy harvesting mechanisms with three, five, two, and six piezoelectric elements, respectively, the techniques described herein can be applied to energy harvesting mechanisms with any suitable number of piezoelectric elements, such as two, three, four, five, six, seven, eight, nine, 10, 15, 20, or more piezoelectric elements. Some or all of the piezoelectric elements can be oriented in different directions to allow for energy harvesting from different directions of input motion, as described herein. For example, some or all of the piezoelectric elements can face different directions so that the respective planes of the piezoelectric elements are offset from each other, such as by an angle greater than or equal to 60°, 70°, 80°, 90°, 100°, 110°, 120°, 130°, 140°, 150, 160°, or 170°. Accordingly, the respective principal bending directions of some or all of the piezoelectric elements can be offset from each other, such as by an angle greater than or equal to 5°, 10°, 20°, 30°, 40°, 50°, 60°, 70°, 80°, 90°, 100°, 110°, or 120°. The piezoelectric elements can be arranged in any suitable configuration, such as a radially symmetric configuration or a radially asymmetric configuration. Some or all of the piezoelectric elements can be spaced apart from each other (e.g., in a radially distributed and/or folded configuration, such as the configurations shown in FIGS. 5A-7B), or some or all of the piezoelectric elements can overlap each other (e.g., in an interleaved and/or coaxial configuration, such as the configuration shown in FIGS. 8A-8E).

In some embodiments, the energy harvesting mechanisms described herein include an odd number of piezoelectric elements. In such embodiments, the piezoelectric elements can be coupled in series, with the distal end portion of the first piezoelectric element coupled to a fixation region within the housing, the proximal end portion of the last piezoelectric element coupled to the harvester mass, and the intermediate piezoelectric elements being coupled to neighboring piezoelectric elements at their respective proximal and distal end portions. For instance, the configurations of piezoelectric elements described with respect to FIGS. 5A-7B can be extended to energy harvesting mechanisms with seven piezoelectric elements, nine piezoelectric elements, eleven piezoelectric elements, etc. In other embodiments, however, some or all of the piezoelectric elements can instead be coupled in parallel, with the respective distal end portions of the piezoelectric elements being coupled to the fixation region, and the respective proximal end portions of the piezoelectric elements being coupled to the harvester mass. This approach can be used, for example, in embodiments where the piezoelectric elements are sufficiently flexible so the resonant frequency of the energy harvesting mechanism remains suitable for harvesting from physiological motion, as described elsewhere herein.

In some embodiments, the energy harvesting mechanisms described herein include an even number of piezoelectric elements. In such embodiments, one or more of the piezoelectric elements can include structures that allow the piezoelectric elements to effectively behave as a set of components coupled in series. For example, any of the piezoelectric elements can include two or more structures that are movable relative to each other to act as a set of fan-folded beams, e.g., as described with respect to FIGS. 8A-8E. Moreover, in some embodiments, the piezoelectric elements can be coupled in series in an end-to-end, unfolded configuration, e.g., as described with respect to FIG. 9. In other embodiments, however, some or all of the piezoelectric elements can instead be coupled in parallel, e.g., if the piezoelectric elements are sufficiently flexible so the resonant frequency of the energy harvesting mechanism remains suitable for harvesting from physiological motion, as described elsewhere herein.

### Examples

The following examples are included to further describe some aspects of the present technology, and should not be used to limit the scope of the technology.
1. A device comprising:
a housing configured to be implanted within a patient, wherein the housing comprises a proximal end, a distal end, and a longitudinal axis extending between the proximal end and the distal end;
a power source positioned within the housing; and
an energy harvesting mechanism positioned within the housing and configured to charge the power source, wherein the energy harvesting mechanism comprises a plurality of piezoelectric elements that are aligned with the longitudinal axis of the housing, and wherein each piezoelectric element faces a different direction.
2. The device of Example 1, wherein the plurality of piezoelectric elements are arranged radially around the longitudinal axis of the housing.
3. The device of Example 1 or 2, wherein the plurality of piezoelectric elements are arranged in a radially symmetric configuration.
4. The device of any one of Examples 1 to 3, wherein each piezoelectric element has a flattened body defining a plane, and wherein the planes of the plurality of piezoelectric elements are offset from each other.
5. The device of any one of Examples 1 to 4, wherein each piezoelectric element has a flattened body defining a plane, and wherein each piezoelectric element faces a different direction by virtue of each such plane facing a different direction.
6. The device of any one of Examples 1 to 5, wherein the energy harvesting mechanism is coupled to the housing at a fixation region, and wherein the energy harvesting mechanism further comprises a harvester mass spaced apart from the fixation region.
7. The device of Example 6, wherein the plurality of piezoelectric elements comprise:
a first piezoelectric element coupled to the fixation region, and
a second piezoelectric element coupled to the harvester mass.
8. The device of Example 7, wherein the plurality of piezoelectric elements comprise at least one additional piezoelectric element coupling the first piezoelectric element to the second piezoelectric element.
9. The device of Example 8, wherein the first piezoelectric element, the second piezoelectric element, and the at least one additional piezoelectric element are coupled to each other in series.
10. The device of Example 7, wherein:
the first piezoelectric element includes a first end portion coupled to the fixation region, and a second end portion opposite the first end portion, and
the second piezoelectric element includes a first end portion coupled to the first end portion of the first piezoelectric element, and a second end portion opposite the first end portion and coupled to the harvester mass.
11. The device of Example 10, wherein the first piezoelectric element comprises a pair of outer members and an inner member between the pair of outer members, the pair of outer members being coupled to the fixation region, and the inner member being coupled to the first end portion of the second piezoelectric element.
12. The device of any one of Examples 1 to 11, wherein each piezoelectric element comprises a piezoelectric bimorph beam.
13. The device of any one of Examples 1 to 12, wherein the housing is configured to be implanted in a heart of the patient, and wherein the device further comprises one or more electrodes configured to deliver electrical stimulation to the heart.
14. The device of any one of Examples 1 to 13, wherein the energy harvesting mechanism comprises a cross-sectional dimension orthogonal to the longitudinal axis of the housing, and the cross-sectional dimension is no more than 10 mm.
15. A device comprising:
a housing configured to be implanted within a patient, wherein the housing comprises a proximal end, a distal end, and a longitudinal axis extending between the proximal end and the distal end;
a power source positioned within the housing; and
an energy harvester positioned within the housing and configured to charge the power source, wherein the energy harvester comprises a plurality of piezoelectric members extending along the longitudinal axis of the housing, and wherein each piezoelectric member has a different principal bending direction.
16. The device of Example 15, wherein the principal bending direction of each piezoelectric member corresponds to a direction of motion that maximizes a power output of the piezoelectric member.
17. The device of Example 15 or 16, wherein the principal bending direction of each piezoelectric member corresponds to a direction of motion that minimizes an amount of force to deflect the piezoelectric member.
18. The device of any one of Examples 15 to 17, wherein each piezoelectric member has a longitudinal axis and a transverse axis, and the principal bending direction of each piezoelectric member is orthogonal to the longitudinal axis and the transverse axis of the piezoelectric member.
19. The device of any one of Examples 15 to 18, wherein the energy harvester is coupled to the housing at a fixation region, and wherein the energy harvester further comprises a harvester mass spaced apart from the fixation region.
20. The device of Example 19, wherein the plurality of piezoelectric members comprise:
a first piezoelectric member coupled to the fixation region, and
a second piezoelectric member coupled to the harvester mass.
21. The device of Example 20, wherein the plurality of piezoelectric members comprise at least one additional piezoelectric member coupling the first piezoelectric member to the second piezoelectric member.
22. The device of Example 21, wherein the first piezoelectric member, the second piezoelectric member, and the at least one additional piezoelectric member are coupled to each other in an end-to-end configuration.
23. The device of Example 20, wherein:
the first piezoelectric member includes a first end portion coupled to the fixation region, and a second end portion opposite the first end portion, and
the second piezoelectric member includes a first end portion coupled to the first end portion of the first piezoelectric member, and a second end portion opposite the first end portion and coupled to the harvester mass.
24 The device of Example 23, wherein the first piezoelectric member comprises an outer structure and an inner structure between the outer structure, the outer structure being coupled to the fixation region, and the inner structure being coupled to the first end portion of the second piezoelectric member.
25. The device of any one of Examples 15 to 24, wherein each piezoelectric member comprises a piezoelectric bimorph beam.
26. The device of any one of Examples 15 to 25, wherein the housing is configured to be implanted in a heart of the patient, and wherein the device further comprises one or more electrodes configured to deliver electrical stimulation to the heart.
27. The device of any one of Examples 15 to 26, wherein the energy harvester comprises a cross-sectional dimension orthogonal to the longitudinal axis of the housing, and the cross-sectional dimension is no more than 10 mm.
28. A device comprising:
a housing configured to be implanted within a patient;
a power source positioned within the housing; and
an energy harvesting mechanism positioned within the housing and configured to charge the power source, wherein the energy harvesting mechanism comprises:
   a plurality of piezoelectric elements, and
   one or more joints mechanically and electrically coupling the plurality of piezoelectric elements to each other in series.
29. The device of Example 28, wherein the plurality of piezoelectric elements are each configured to produce a current in response to motion of the device, and wherein the one or more joints are each configured to transmit the current from one of the piezoelectric elements to another one of the piezoelectric elements.
30. The device of Example 28 or 29, wherein the energy harvesting mechanism is coupled to the housing at a fixation region, and wherein the energy harvesting mechanism further comprises a harvester mass spaced apart from the fixation region.
31. The device of Example 30, wherein the plurality of piezoelectric elements comprise:
a first piezoelectric element coupled to the fixation region,
a second piezoelectric element coupled to the harvester mass, and
a third piezoelectric element coupled in series between the first piezoelectric element and the second piezoelectric element.
32. The device of Example 31, wherein the one or more joints comprise:
a first joint mechanically and electrically coupling the first piezoelectric element to the third piezoelectric element, and
a second joint mechanically and electrically coupling the second piezoelectric element to the third piezoelectric element.
33. The device of Example 31, wherein:
the plurality of piezoelectric elements comprise a fourth piezoelectric element coupled in series between the first piezoelectric element and the second piezoelectric element, and
the one or more joints comprises:
   a first joint mechanically and electrically coupling the first piezoelectric element to the third piezoelectric element, and
   a second joint mechanically and electrically coupling the second piezoelectric element to the fourth piezoelectric element.
34. The device of any one of Examples 28 to 33, wherein the one or more joints are each made from a combination of a conductive material and an insulative material.
35. The device of any one of Examples 28 to 33, wherein the one or more joints are each made entirely from a conductive material.
36. The device of any one of Examples 28 to 35, wherein the one or more joints are rigid.
37. The device of any one of Examples 28 to 35, wherein the one or more joints are flexible.
38. The device of any one of Examples 28 to 37, wherein each piezoelectric element faces a different direction.
39. The device of any one of Examples 28 to 38, wherein each piezoelectric element has a different principal bending direction.

### CONCLUSION

Although many of the embodiments are described above with respect to systems, devices, and methods for cardiac pacing, the technology is applicable to other applications and/or other approaches, such as other therapies involving implantable devices. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1-9.

The embodiments of the present technology can be implemented, at least in part, in hardware, software, firmware or any combination thereof. For example, various embodiments can be implemented within one or more processors, including one or more microprocessors, DSPs, ASICs, FPGAs, or any other equivalent integrated or discrete logic circuitry, as well as any combinations of such components, embodied in programmers (e.g., physician or patient programmers), stimulators, or other devices. The terms “processor” or “processing circuitry” may generally refer to any of the foregoing logic circuitry, alone or in combination with other logic circuitry, or any other equivalent circuitry.

The various processes described herein can be partially or fully implemented using program code including instructions executable by one or more processors of a computing system for implementing specific logical functions or steps in the process. The program code can be stored on any type of computer-readable medium, such as a storage device including a disk or hard drive. Computer-readable media containing code, or portions of code, can include any appropriate media known in the art, such as non-transitory computer-readable storage media. Computer-readable media can include volatile and non-volatile, removable and non-removable media implemented in any method or technology for storage and/or transmission of information, including, but not limited to, random-access memory (RAM), read-only memory (ROM), electrically erasable programmable read-only memory (EEPROM), flash memory, or other memory technology; compact disc read-only memory (CD-ROM), digital video disc (DVD), or other optical storage; magnetic cassettes, magnetic tape, magnetic disk storage, or other magnetic storage devices; solid state drives (SSD) or other solid state storage devices; or any other medium which can be used to store the desired information and which can be accessed by a system device.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent variations in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded.

To the extent any materials incorporated herein by reference conflict with the present disclosure, the present disclosure controls.

It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1.-15. (canceled)

16. A device comprising:
a housing configured to be implanted within a patient, wherein the housing comprises a proximal end, a distal end, and a longitudinal axis extending between the proximal end and the distal end;
a power source positioned within the housing; and
an energy harvesting mechanism positioned within the housing and configured to charge the power source, wherein the energy harvesting mechanism comprises a plurality of piezoelectric elements that are aligned with the longitudinal axis of the housing, and wherein each piezoelectric element faces a different direction.

17. The device of claim 16, wherein the plurality of piezoelectric elements are arranged radially around the longitudinal axis of the housing.

18. The device of claim 16, wherein the plurality of piezoelectric elements are arranged in a radially symmetric configuration.

19. The device of claim 16, wherein each piezoelectric element has a flattened body defining a plane, and wherein the planes of the plurality of piezoelectric elements are offset from each other.

20. The device of claim 16, wherein each piezoelectric element has a flattened body defining a plane, and wherein each piezoelectric element faces a different direction by virtue of each such plane facing a different direction.

21. The device of claim 16, wherein the energy harvesting mechanism is coupled to the housing at a fixation region, and wherein the energy harvesting mechanism further comprises a harvester mass spaced apart from the fixation region.

22. The device of claim 21, wherein the plurality of piezoelectric elements comprise:
a first piezoelectric element coupled to the fixation region, and
a second piezoelectric element coupled to the harvester mass.

23. The device of claim 22, wherein the plurality of piezoelectric elements comprise at least one additional piezoelectric element coupling the first piezoelectric element to the second piezoelectric element.

24. The device of claim 23, wherein the first piezoelectric element, the second piezoelectric element, and the at least one additional piezoelectric element are coupled to each other in series.

25. The device of claim 16 wherein the housing is configured to be implanted in a heart of the patient, and wherein the device further comprises one or more electrodes configured to deliver electrical stimulation to the heart.

26. A device comprising:
a housing configured to be implanted within a patient, wherein the housing comprises a proximal end, a distal end, and a longitudinal axis extending between the proximal end and the distal end;
a power source positioned within the housing; and
an energy harvester positioned within the housing and configured to charge the power source, wherein the energy harvester comprises a plurality of piezoelectric members extending along the longitudinal axis of the housing, and wherein each piezoelectric member has a different principal bending direction.

27. The device of claim 26, wherein the principal bending direction of each piezoelectric member corresponds to a direction of motion that maximizes a power output of the piezoelectric member.

28. The device of claim 26, wherein the principal bending direction of each piezoelectric member corresponds to a direction of motion that minimizes an amount of force to deflect the piezoelectric member.

29. The device of claim 26, wherein each piezoelectric member has a longitudinal axis and a transverse axis, and the principal bending direction of each piezoelectric member is orthogonal to the longitudinal axis and the transverse axis of the piezoelectric member.

30. The device of claim 26, wherein the energy harvester is coupled to the housing at a fixation region, and wherein the energy harvester further comprises a harvester mass spaced apart from the fixation region.

31. The device of claim 30, wherein the plurality of piezoelectric members comprise:
a first piezoelectric member coupled to the fixation region, and
a second piezoelectric member coupled to the harvester mass.

32. The device of claim 31, wherein the plurality of piezoelectric members comprise at least one additional piezoelectric member coupling the first piezoelectric member to the second piezoelectric member.

33. The device of claim 32, wherein the first piezoelectric member, the second piezoelectric member, and the at least one additional piezoelectric member are coupled to each other in an end-to-end configuration.

34. The device of claim 31, wherein:
the first piezoelectric member includes a first end portion coupled to the fixation region, and a second end portion opposite the first end portion, and
the second piezoelectric member includes a first end portion coupled to the first end portion of the first piezoelectric member, and a second end portion opposite the first end portion and coupled to the harvester mass.

35. The device of claim 26, wherein the housing is configured to be implanted in a heart of the patient, and wherein the device further comprises one or more electrodes configured to deliver electrical stimulation to the heart.
